(19) United States
(12) Patent Application Publication
(10) Pub. No.: US 2026/0269064 A1
(43) Pub. Date: Sep. 10, 2026

(54) CLOSED-LOOP PSYCHIATRIC CLINICAL DECISION SUPPORT USING PROBABILISTIC STATE ESTIMATION AND SAFETY-CONSTRAINED POLICY COMPUTATION

(71) Applicant: WONDER SCIENCES, INC., Marina del Rey, CA (US)

(72) Inventor: Ryan R. Magnussen, Marina del Rey, CA (US)

(21) Appl. No.: 19/559,179

(22) Filed: Mar. 6, 2026

Related U.S. Application Data
(60) Provisional application No. 63/768,816, filed on Mar. 7, 2025.

(51) Int. Cl. G16H 50/20; G16H 20/00; G16H 50/30

(52) U.S. Cl. CPC G16H 50/20; G16H 20/00; G16H 50/30

(57) ABSTRACT

A computer-implemented system, method, and non-transitory computer-readable medium for clinician-governed, uncertainty-bounded, closed-loop psychiatric clinical decision support. The system receives multimodal longitudinal patient data, transforms it into a unified temporally indexed tensor representation, and generates probabilistic state vectors using trait-state decomposition and hierarchical latent variable models. Epistemic and aleatoric uncertainty are separately tracked and propagated through all downstream stages. The system computes safety-constrained policy outputs via bounded optimization subject to guardrails, wherein increased uncertainty algorithmically shrinks the feasible action space. Clinician-review artifacts with rationale traces are generated. The system compiles approved outputs into executable workflow artifacts with adherence-aware pacing. The system causes execution of the clinician-approved treatment plan to deliver treatment to the patient.

### CROSS-REFERENCE TO RELATED APPLICATIONS

The present application claims the benefit of and priority to U.S. Provisional Application No. 63/768,816 filed on March 7, 2025, which is incorporated by reference.

### BACKGROUND

Psychiatrists and psychotherapists are important roles in supporting mental health in society. These healthcare providers can analyze in-person sessions with a patient and other electronic health record (EHR) data in diagnosing mental health issues in individuals. These assessments can lead the healthcare providers to prescribe various treatments, including medication.

The diagnosis can involve the synthesis of heterogeneous, asynchronous, and frequently discordant clinical information—including structured rating scales, clinician-entered assessments, patient-reported outcome measures, behavioral engagement signals, and, in certain implementations, physiological telemetry—into clinical judgments that balance therapeutic benefit against destabilization risk across multiple time horizons. The complexity of this synthesis is compounded by high rates of psychiatric comorbidity, nonlinear interactions among symptom dimensions, variable treatment-response latencies, and the safety-critical nature of decisions involving suicide risk, pharmacologic intervention, and crisis escalation. Computerized clinical decision support (CDS) systems have been developed to assist clinicians in managing aspects of this complexity.

Certain existing CDS systems for mental healthcare evaluate patient status by comparing observed measurement scores against population-derived thresholds. In one illustrative example, a standardized depression screening instrument may classify a score at or above a fixed cutoff as indicating a particular severity category, without reference to the individual patient’s historical trajectory. In such an approach, a patient whose chronic baseline score is already elevated may experience clinically significant acute deterioration that the system does not detect because the absolute score does not cross a higher threshold. Conversely, a patient whose stable, well-managed baseline exceeds a population threshold may generate persistent alerts that carry no acute clinical significance. These characteristics arise where the system does not model deviation from the individual patient’s own baseline trajectory, and may contribute to reduced alert specificity in clinical deployment.

Certain existing CDS systems for psychiatric assessment produce categorical, deterministic outputs—for example, a binary classification of a diagnostic condition as present or absent. Such systems do not provide a mechanism for the clinician to distinguish between a classification supported by abundant, concordant evidence and a classification derived from sparse, conflicting, or ambiguous data. When evidence is insufficient, such a system may either withhold output or produce a point estimate that does not communicate the degree of evidentiary support underlying the classification. In psychiatric practice, where diagnostic uncertainty is common and comorbid presentations are frequent, the absence of calibrated uncertainty information may limit the clinical utility of system outputs.

Furthermore, certain systems that do produce confidence scores do not distinguish between epistemic uncertainty—arising from insufficient data and reducible through additional observation—and aleatoric uncertainty—arising from inherent stochastic variability in the patient’s condition and not reducible through additional data collection. Where these uncertainty types are not separately identified and propagated, downstream computations cannot distinguish between situations in which additional data collection would resolve ambiguity and situations in which the ambiguity is intrinsic.

### SUMMARY

According to an aspect of the invention, a computer-implemented system comprises one or more processors and non-transitory memory storing instructions that, when executed, cause the processors to implement an Adaptive Neuro-AI Framework (ANAF) engine that continuously constructs and updates five linked probabilistic state vectors encoding a patient's psychiatric status across diagnostic, care, risk, pharmacologic, and operational dimensions, wherein the ANAF engine receives multimodal longitudinal patient data transformed into a unified temporally indexed tensor representation and performs individualized prior construction, trait-state decomposition separating enduring patient characteristics from acute clinical phenomena, Bayesian state updates incorporating new observations with confidence-weighted likelihood functions, longitudinal drift and volatility analytics computing velocity, acceleration, and volatility fingerprints across state dimensions, uncertainty propagation decomposing and propagating epistemic and aleatoric uncertainty components through all downstream computations, and conservative gating that applies one or more conservative actions when propagated uncertainty exceeds configurable thresholds.

The ANAF engine provides technical advantages through its adaptive calibration architecture, wherein the engine constructs individualized prior distributions for each patient by fitting distributional models to the patient's own historical state trajectory when sufficient data is available, combining limited patient data with demographically stratified population priors when historical data is insufficient, or initializing with population-level priors having inflated covariance when no historical data is available such that initial conservatism is proportional to the system's uncertainty about the specific patient, and wherein the trait-state decomposition enables the engine to distinguish chronic baseline features from actionable acute changes by modeling observed signals as the sum of slowly varying trait components updated on longer timescales with higher regularization and rapidly varying state components updated at each observation with lower regularization, such that a patient whose chronic baseline symptom level is elevated can have clinically significant acute deterioration detected relative to their own trajectory rather than being masked by population-threshold comparisons, and such that a patient whose stable baseline exceeds population norms does not generate persistent alerts carrying no acute clinical significance.

The ANAF engine operates under clinician governance as a decision-support tool rather than an autonomous clinical agent, wherein the engine generates clinician-review artifacts comprising recommended actions, rationale traces identifying the state-vector dimensions and data sources contributing to each recommendation, constraint-satisfaction summaries documenting which safety constraints were evaluated and satisfied, and uncertainty annotations distinguishing epistemic uncertainty that could be reduced through additional data collection from aleatoric uncertainty that is intrinsic and must be managed through conservative action, and wherein no computational output constitutes a final clinical action without explicit clinician authorization such that no diagnosis is rendered, no medication is prescribed, modified, or discontinued, and no crisis-escalation protocol is activated without clinician approval, and wherein the safety-constrained workflow is enforced through hard constraints including contraindication exclusion, drug-drug interaction restrictions, destabilization-risk gating that restricts treatment and medication intensification when the Risk State Vector indicates elevated risk, cognitive-load budgeting that prevents over-prescription of concurrent therapeutic tasks, and uncertainty-driven action-space shrinking that progressively restricts the feasible action space as epistemic uncertainty increases such that under maximal uncertainty only maintenance and increased-monitoring actions remain feasible.

Clause 1. A computer-implemented method for clinician-governed, uncertainty-bounded, closed-loop psychiatric clinical decision support, the computer-implemented method executed by one or more processors and comprising: accessing multimodal patient data for a patient from a plurality of data sources; generating a tensor representation synthesizing the multimodal data into a unified temporally indexed structure comprising, for each observation, feature vectors, missingness encodings, confidence weights, source provenance metadata, and consent-gating flags; generating one or more probabilistic state vectors encoding psychiatric status across a plurality of clinical dimensions, wherein generating the one or more probabilistic state vectors comprises decomposing the multimodal patient data using a hierarchical latent variable model, and computing, for each state vector, mean estimates and associated uncertainty parameters; computing a longitudinal trajectory over the one or more probabilistic state vectors, the longitudinal trajectory comprising at least one of: drift, acceleration, and volatility estimates for each state-vector dimension; generating a treatment plan through safety-constrained optimization on the longitudinal trajectory, wherein the optimization is subject to hard safety constraints comprising at least one of contraindication exclusion, destabilization-risk gating, or cognitive-load budgeting, wherein increased uncertainty in the one or more probabilistic state vectors algorithmically shrinks action space of the optimization; causing presentation of the treatment plan on a clinician-facing user interface to obtain clinician approval; receiving, via the clinician-facing user interface, clinician approval of the treatment plan; upon receipt of clinician approval, causing execution of the treatment plan by presenting one or more actions of the treatment plan on a patient-facing user interface.

Clause 2. The computer-implemented method of clause 1, wherein the plurality of data sources comprises electronic health records, clinician-entered assessments, patient-reported outcome measures, behavioral engagement telemetry, biometric sensor streams, pharmacy and medication data, administrative and operational data, or some combination thereof.

Clause 3. The computer-implemented method of clause 1, wherein generating the one or more probabilistic state vectors: comprises constructing individualized prior distributions by: fitting a distributional model to a state trajectory derived from historical patient data.

Clause 4. The computer-implemented method of clause 1, wherein generating the one or more probabilistic state vectors: comprises constructing individualized prior distributions by: initializing with a population-level prior having a covariance inflated by a configurable factor to reflect uncertainty about the patient.

Clause 5. The computer-implemented method of clause 1, wherein generating the one or more probabilistic state vectors further comprises performing Bayesian state updates upon receipt of new data, wherein a posterior distribution is computed as proportional to a product of a prior distribution and an observation likelihood, and wherein the observation likelihood is modulated by confidence weights such that higher-confidence observations contribute more strongly to the posterior update.

Clause 6. The computer-implemented method of clause 5, wherein features with indicators representing that the feature was not observed are excluded from the observation likelihood computation, such that dimensions associated with missing features have their uncertainty increase over time through prior dynamics alone.

Clause 7. The computer-implemented method of clause 1, wherein computing longitudinal trajectory statistics further comprises: computing volatility fingerprints, each volatility fingerprint comprising a pattern of variance changes across a plurality of state-vector dimensions; and comparing said volatility fingerprints against a library of learned destabilization signatures.

Clause 8. The computer-implemented method of clause 1, wherein computing the longitudinal trajectory further comprises: applying Bayesian online change-point detection that maintains a posterior distribution over time since a recent change point and flags a dimension as having undergone a statistically significant shift when a posterior probability of the recent change point exceeds a configurable threshold.

Clause 9. The computer-implemented method of clause 1, further comprising: computing safety-constrained policy outputs by applying conservative gating when propagated uncertainty exceeds configurable thresholds, said conservative gating comprising at least one of: reducing a magnitude of recommended changes, increasing recommended monitoring frequency, narrowing a set of permissible actions, withholding output pending clinician review, and annotating the output with an explicit low-confidence indicator identifying whether elevated uncertainty is primarily epistemic or aleatoric.

Clause 10. The computer-implemented method of clause 1, wherein generating the one or more probabilistic state vectors is performed using an inference method selected from the group consisting of: variational inference with a parametric posterior approximation; sequential Monte Carlo with weighted particle representation; extended Kalman filtering adapted for psychiatric state spaces; and unscented Kalman filtering.

Clause 11. The computer-implemented method of clause 1, wherein the unified temporally indexed representation is implemented as a sparse data structure storing observed entries, configured for incremental update upon receipt of new data without full re-computation and deterministic reconstruction of any prior data state from logged observations.

Clause 12. The computer-implemented method of clause 1, further comprising: evaluating concordance across data modalities for each clinical construct represented in the state vectors, and when statistically significant discordance is detected between modalities, increasing the uncertainty parameters of affected state-vector dimensions.

Clause 13. The computer-implemented method of clause 12, wherein clinician-review artifacts include discordance annotations identifying which modalities disagree and a magnitude of disagreement for each affected clinical construct.

Clause 14. The computer-implemented method of clause 1, wherein further comprising: compiling outputs into operationally executable workflow artifacts.

Clause 15. The computer-implemented method of clause 14, wherein compiling the outputs into operationally executable workflow artifacts further comprises routing the patient among a plurality of care tiers modeled as a directed graph.

Clause 16. The computer-implemented method of clause 14, wherein compiling the outputs into operationally executable workflow artifacts further comprises decomposing a clinician-approved care plan into a directed acyclic graph of atomic executable tasks, each task comprising a task type and a scheduled execution time or trigger condition.

Clause 17. The computer-implemented method of clause 1, further comprising: executing a crisis response protocol modeled as a state machine comprising a sequence of escalation states with transition rules, wherein each state transition carries a service-level-agreement timer.

Clause 18. The computer-implemented method of clause 1, wherein the one or more probabilistic state vectors include a diagnostic state vector, and wherein generating the diagnostic state vector comprises: encoding psychiatric diagnostic criteria as a probabilistic constraint graph in which each criterion is associated with an activation probability derived from relevant state-vector dimensions; and computing a posterior probability distribution over the number of criteria to yield a graded diagnostic confidence.

Clause 19. The computer-implemented method of clause 1, wherein the one or more probabilistic state vectors include a risk state vector, and wherein generating the risk state vector comprises: computing multi-horizon probabilistic risk trajectories by projecting a risk state vector forward through a learned state-transition model, the trajectories comprising probability distributions over future risk states at a plurality of time horizons.

Clause 20. The computer-implemented method of clause 1, wherein the one or more probabilistic state vectors include a latent pharmacologic state vector, and wherein generating the latent pharmacologic state vector comprises: modeling the latent pharmacologic state vector against stability-flexibility balance, adverse-event susceptibility, tolerance dynamics, or some combination thereof as time-varying probabilistic estimates.

Clause 21. The computer-implemented method of clause 1, wherein generating the treatment plan comprises: selecting a set of candidate medication actions from a parameterized control-action library; and applying constrained optimization subject to hard constraints, with uncertainty-driven action-space shrinking that restricts the set of candidate medication actions as uncertainty parameters increase.

Clause 22. The computer-implemented method of clause 21, wherein the uncertainty-driven action-space shrinking defines an uncertainty-adjusted action radius that is a monotonically decreasing function of epistemic uncertainty.

Clause 23. The computer-implemented method of clause 21, wherein generating the treatment plan further comprises: identifying high-sensitivity therapeutic windows from the longitudinal trajectory; and restricting medication adjustments during the high-sensitivity therapeutic windows.

Clause 24. The computer-implemented method of clause 1, wherein the one or more probabilistic state vectors comprise a plurality of linked state vectors across distinct clinical domains, and wherein the method further comprises: generating a cross-pillar coupling matrix defining directional relationships between state vectors, such that a change in a source state vector causes conditional prior updates in coupled target state vectors.

Clause 25. The computer-implemented method of clause 24, wherein the cross-pillar coupling matrix couples elevated destabilization risk in a risk state vector with restriction in permissible treatment intensity in a treatment sequencing computation or with restriction in permissible medication action space in a pharmacologic parameter computation.

Clause 26. The computer-implemented method of clause 24, wherein the cross-pillar coupling matrix couples increased diagnostic uncertainty in a diagnostic state vector with decreases in permissible medication action space in a pharmacologic parameter computation.

Clause 27. The computer-implemented method of clause 24, wherein the cross-pillar coupling matrix couples medication volatility indicators in a pharmacologic state vector feed back to a risk state vector as input features.

Clause 28. The computer-implemented method of clause 24, wherein the cross-pillar coupling matrix couples operational feasibility constraints in an operational state vector to bound treatment and medication recommendations.

Clause 29. A non-transitory computer-readable storage medium storing instructions for clinician-governed, uncertainty-bounded, closed-loop psychiatric clinical decision support, the instructions that, when executed, cause one or more processors to perform operations comprising: accessing multimodal patient data for a patient from a plurality of data sources; generating a tensor representation synthesizing the multimodal data into a unified temporally indexed structure comprising, for each observation, feature vectors, missingness encodings, confidence weights, source provenance metadata, and consent-gating flags; generating one or more probabilistic state vectors encoding psychiatric status across a plurality of clinical dimensions, wherein generating the one or more probabilistic state vectors comprises decomposing the multimodal patient data using a hierarchical latent variable model, and computing, for each state vector, mean estimates and associated uncertainty parameters; computing a longitudinal trajectory over the probabilistic state vectors, the longitudinal trajectory comprising at least one of: drift, acceleration, and volatility estimates for each state-vector dimension; generating a treatment plan through safety-constrained optimization on the longitudinal trajectory, wherein the optimization is subject to hard safety constraints comprising at least one of contraindication exclusion, destabilization-risk gating, or cognitive-load budgeting, wherein increased uncertainty in the one or more probabilistic state vectors algorithmically shrinks action space of the optimization; causing presentation of the treatment plan on a clinician-facing user interface to obtain clinician approval; receiving, via the clinician-facing user interface, clinician approval of the treatment plan; upon receipt of clinician approval, causing execution of the treatment plan by presenting one or more actions of the treatment plan on a patient-facing user interface.

Clause 30. A system for clinician-governed, uncertainty-bounded, closed-loop psychiatric clinical decision support, the system comprising: one or more processors; and a non-transitory computer-readable storage medium storing instructions that, when executed, cause the one or more processors to perform operations comprising: accessing multimodal patient data for a patient from a plurality of data sources; generating a tensor representation synthesizing the multimodal data into a unified temporally indexed structure comprising, for each observation, feature vectors, missingness encodings, confidence weights, source provenance metadata, and consent-gating flags; generating one or more probabilistic state vectors encoding psychiatric status across a plurality of clinical dimensions, wherein generating the one or more probabilistic state vectors comprises decomposing the multimodal patient data using a hierarchical latent variable model, and computing, for each state vector, mean estimates and associated uncertainty parameters; computing a longitudinal trajectory over the probabilistic state vectors, the longitudinal trajectory comprising at least one of: drift, acceleration, and volatility estimates for each state-vector dimension; generating a treatment plan through safety-constrained optimization on the longitudinal trajectory, wherein the optimization is subject to hard safety constraints comprising at least one of contraindication exclusion, destabilization-risk gating, or cognitive-load budgeting, wherein increased uncertainty in the one or more probabilistic state vectors algorithmically shrinks action space of the optimization; causing presentation of the treatment plan on a clinician-facing user interface to obtain clinician approval; receiving, via the clinician-facing user interface, clinician approval of the treatment plan; upon receipt of clinician approval, causing execution of the treatment plan by presenting one or more actions of the treatment plan on a patient-facing user interface.

Clause 31. A computer-implemented method for efficient representation and incremental processing of asynchronous multimodal longitudinal data, the method executed by one or more processors and comprising: receiving, from a plurality of heterogeneous data sources at asynchronous observation times, multimodal data elements, each data element comprising one or more features, a timestamp, and a source identifier; transforming each received data element into a unified temporally indexed tensor representation implemented as a sparse data structure that stores entries only for observed features at observed times, wherein each entry comprises a feature value, a missingness indicator encoding which features were not observed, a confidence weight quantifying reliability of the feature value, and a provenance metadata tag linking the feature value to its originating data source and extraction method; upon receipt of a new data element having k features, performing an incremental update of the tensor representation in time proportional to k without re-computation over a full temporal history of prior observations; maintaining a sparse observation log from which any prior state of the tensor representation is deterministically reconstructable; and generating, from the tensor representation, one or more probabilistic state estimates by performing inference that weights each feature's contribution according to its associated confidence weight and excludes features having missingness indicators from the inference computation such that unobserved features do not artificially constrain the resulting state estimates.

Clause 32. The computer-implemented method of clause 31, wherein the multimodal data elements comprise clinical data from electronic health records, patient-reported outcome measures, behavioral engagement telemetry, biometric sensor streams, or some combination thereof.

Clause 33. The computer-implemented method of clause 31, wherein the sparse data structure provides memory consumption proportional to a number of actual observations rather than a product of a number of data sources, a number of time points, and a number of features.

Clause 34. The computer-implemented method of clause 31, wherein each entry in the sparse observation log is assigned a cryptographic timestamp generated using a hash-chain mechanism incorporating a hash of a prior timestamp, creating a tamper-evident sequence.

Clause 35. A computer-implemented method for cross-domain probabilistic state estimation with coupled uncertainty propagation, the method executed by one or more processors and comprising: maintaining a plurality of probabilistic state vectors, each state vector encoding a patient's estimated status within a distinct clinical domain and each state vector comprising a mean estimate vector, a covariance matrix, an epistemic uncertainty vector representing uncertainty attributable to insufficient data, and an aleatoric variance vector representing uncertainty attributable to inherent stochastic variability; storing a coupling matrix defining, for each ordered pair of state vectors, a directional conditional-prior update relationship specifying how changes in a source state vector constrain a target state vector; upon update of a source state vector, propagating constrained effects to each coupled target state vector by modifying the target state vector's prior distribution according to the coupling matrix, wherein an increase in uncertainty in the source state vector produces corresponding constraint tightening in the coupled target state vector; and generating a clinical decision-support output from the plurality of coupled state vectors, wherein the decision-support output reflects cross-domain interactions enforced by the coupling matrix.

Clause 36. The computer-implemented method of clause 35, wherein the plurality of probabilistic state vectors comprises a diagnostic state vector, a care state vector, a risk state vector, a pharmacologic state vector, and an operational delivery state vector.

Clause 37. The computer-implemented method of clause 35, wherein the coupling matrix couples elevated destabilization risk in a risk state vector with restriction of permissible treatment intensity in a treatment sequencing computation and with restriction of permissible medication action space in a pharmacologic parameter computation.

Clause 38. The computer-implemented method of clause 35, wherein the coupling matrix couples medication volatility indicators in a pharmacologic state vector back to a risk state vector as input features, such that pharmacologic instability is reflected in destabilization risk forecasts.

Clause 39. The computer-implemented method of clause 35, wherein propagated uncertainty from the plurality of state vectors produces emergent system-wide conservatism without a global conservatism parameter, such that the system autonomously becomes more conservative as aggregate uncertainty across domains increases.

Clause 40. A computer-implemented method for uncertainty-bounded clinical intervention optimization, the method executed by one or more processors and comprising: maintaining one or more probabilistic state vectors encoding a patient's clinical status, each state vector comprising a mean estimate and an epistemic uncertainty parameter representing uncertainty reducible through additional data collection; defining a parameterized action space comprising a plurality of candidate clinical interventions, each candidate intervention associated with an expected impact magnitude; computing an uncertainty-adjusted action radius as a monotonically decreasing function of the epistemic uncertainty parameter, such that the action radius decreases as epistemic uncertainty increases; restricting the parameterized action space to a feasible action subset comprising only those candidate interventions whose expected impact magnitude does not exceed the uncertainty-adjusted action radius; under a condition of maximal epistemic uncertainty, restricting the feasible action subset to maintenance of a current clinical regimen and increased monitoring actions; and generating a clinician-review artifact presenting the feasible action subset with an annotation indicating the current uncertainty-adjusted action radius and whether the radius restriction is driven by epistemic uncertainty or aleatoric variance.

Clause 41. A computer-implemented method for detection of clinical destabilization using multi-dimensional volatility pattern matching, the method executed by one or more processors and comprising: computing, for each of a plurality of state-vector dimensions characterizing a patient's clinical status, a volatility estimate representing variance of the dimension over a configurable time window; constructing a volatility fingerprint comprising a vector of the computed volatility estimates across the plurality of state-vector dimensions, the volatility fingerprint encoding a pattern of concurrent volatility changes; maintaining a library of learned destabilization signatures, each signature comprising a reference volatility pattern derived from historical patient data in which the reference pattern preceded a clinically verified destabilization event; comparing the constructed volatility fingerprint against each destabilization signature in the library by computing a similarity score; when the similarity score for at least one destabilization signature exceeds a configurable similarity threshold, generating a destabilization alert comprising a priority indicator, an identification of which destabilization signature was matched, and an attribution trace identifying which state-vector dimensions are contributing most to the volatility pattern; and transmitting the destabilization alert to a clinician-facing interface for acknowledgment.

Clause 42. A computer-implemented method for individualized clinical baseline calibration using hierarchical signal decomposition, the method executed by one or more processors and comprising: receiving a longitudinal sequence of clinical observations for a patient, each observation comprising one or more measured features; decomposing the longitudinal sequence using a hierarchical latent variable model into a slowly varying trait component representing enduring characteristics of the patient and a rapidly varying state component representing acute clinical phenomena, wherein the trait component is updated on longer timescales with higher regularization and the state component is updated at each observation with lower regularization; detecting a clinically significant acute change when a deviation of the state component from the trait component exceeds a configurable threshold, the detection being relative to the individual patient's own trait trajectory rather than relative to a fixed population-derived threshold; and generating an alert indicating the detected acute change, the alert comprising the magnitude of the deviation, the state-vector dimensions in which the deviation was detected, and a comparison showing that the deviation would not have been detected by a population-threshold comparison.

Clause 43. The computer-implemented method of clause 42, wherein a patient whose chronic baseline symptom level is elevated has clinically significant acute deterioration detected relative to the patient's own trait trajectory, even when an absolute score does not cross a population-derived severity threshold.

Clause 44. A computer-implemented method for cross-modality discordance detection with uncertainty-adaptive state estimation, the method executed by one or more processors and comprising: receiving clinical data from a plurality of data modalities, each modality producing a modality-specific estimate for one or more clinical constructs; computing a discordance matrix in which each entry quantifies a degree of disagreement between a pair of modalities for a specific clinical construct, the degree of disagreement computed as a variance of modality-specific estimates normalized by an expected variance under concordance; when a discordance score for a clinical construct significantly exceeds unity, indicating that the modalities are producing inconsistent evidence: inflating an epistemic uncertainty parameter of state-vector dimensions associated with the discordant clinical construct, and annotating a clinician-facing output with a discordance indicator identifying which modalities disagree and a magnitude of the disagreement, without resolving the discordance by selecting one modality's evidence over another or by averaging the discordant estimates; and treating the discordance itself as clinically informative evidence to be investigated by a clinician.

### BRIEF DESCRIPTION OF THE DRAWINGS

FIG. 1 shows a networking environment for an analytics system deploying an adaptive neuro-AI framework engine for adaptive pharmacology tailoring, according to one or more embodiments.

FIG. 2 is a block diagram showing an architecture of the analytics system, according to one or more embodiments.

FIG. 3 is an illustrative workflow of the ANAF engine, according to one or more embodiments.

FIG. 4 shows a schematic diagram of state vector dependencies across the computational pillars of the ANAF engine, according to one or more embodiments.

FIG. 5 shows a block diagram of the Operational Delivery Pillar, according to one or more embodiments.

FIG. 6 shows a block diagram of the Diagnostic State Estimation Pillar, according to one or more embodiments.

FIG. 7 shows a block diagram of the Treatment Sequencing Pillar, according to one or more embodiments.

FIG. 8 shows a block diagram of the Risk Forecasting Pillar, according to one or more embodiments.

FIG. 9 shows a block diagram of the Medication Parameter Modeling Pillar, according to one or more embodiments.

FIG. 10 is a flowchart of adaptive pharmacological treatment delivery, according to one or more embodiments.

FIG. 11 shows a block diagram of a neural network model, according to one or more embodiments.

FIG. 12 is a conceptual diagram of functional blocks of a transformer-based neural network model 1200, according to one or more embodiments.

The figures and flowcharts presented herein are illustrative of representative embodiments and are not intended to limit the scope of the invention to the specific configurations, arrangements, or sequences depicted. A person having ordinary skill in the art, upon review of the present disclosure, would readily understand the underlying principles governing the illustrated architectures and would be capable of implementing variations, modifications, and alternative embodiments that achieve the same functional results without departing from the scope of the invention as defined by the appended claims.

### DETAILED DESCRIPTION

### Overview

The present disclosure provides systems, methods, and non-transitory computer-readable media for closed-loop psychiatric clinical decision support using probabilistic state estimation and safety-constrained computation.

As a technological improvement to the traditional psychiatric assessment paradigm, the system maintains up-to-date state estimations for informing trajectory of the patient over time. This prevents session-to-session infrequency from creating any blind spots in the treatment of the patient.

### Networking Environment

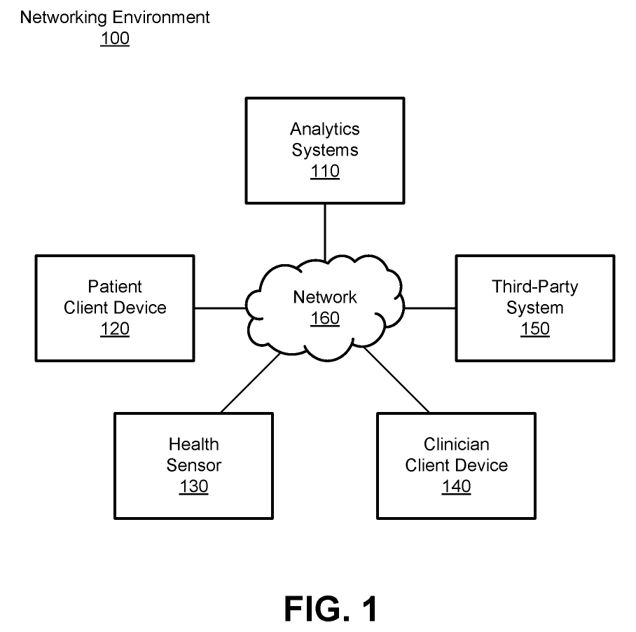

FIG. 1 shows a networking environment for an analytics system deploying an adaptive neuro-AI framework engine for adaptive pharmacology tailoring, according to one or more embodiments.

As shown in FIG. 1, the networking environment 100 includes an analytics system 110, a patient client device 120, one or more health sensors 130, a clinician client device 140, and a third-party system 150 communicating over a network 160.

The analytics system 110 performs computational analyses with the reasoning engine to provide outputs to the client device 120. The analytics system 110 receives signal streams from the client device 120, the health sensor 130, the third-party system 140, or some combination thereof. The analytics system 110 applies the reasoning engine to the received signal streams to determine an output based on the normalized intent representation, retrieved knowledge subgraphs, multi-framework evaluation, constraint enforcement, conflict quantification, uncertainty propagation, or some combination thereof. The analytics system 110 provides the output to the client device 120, and may also provide outputs or audit records to the third-party system 140 or other connected components in the networking environment 100.

The client device 120 presents a user interface for the user to communicate with the analytics system 110. The client device 120 may be a mobile device, a wearable device, another computing system, or some combination thereof. The client device 120 may include one or more input devices, one or more output devices, or some combination thereof. For example, the input devices may include a touchscreen, a text input window, a microphone, other mechanical inputs, other audio inputs, or some combination thereof. For example, the output devices may include an electronic display, a speaker, a haptic assembly, or some combination thereof. As the client device 120 receives data from the analytics system 110, the client device 120 may output the data for presentation to the user via the one or more output devices.

The health sensor 130 captures biometric signals characterizing a user's health state. In one or more embodiments, the health sensor 130 may be implemented on the client device 120. The health sensor may capture biometric signals in conjunction with user input via the client device 120.

In one or more embodiments, the health sensor 130 captures heart rate or derivative signals. The health sensor 130 may include one or more electrodes for measuring heart-related signals, including electrocardiogram (EKG) signals, heart rate, heart rate variability, or other cardiac metrics.

In one or more embodiments, the health sensor 130 captures respiratory cadence signals. The health sensor 130 may include one or more sensors for measuring respiratory patterns, including breath cycle duration, inhale-exhale symmetry, respiratory coherence, or other respiratory metrics.

In one or more embodiments, the health sensor 130 captures electroencephalography (EEG) signals. The health sensor 130 may include one or more electrodes for measuring neural oscillation features, including alpha, theta, beta, or gamma frequency bands, neural synchrony patterns, or other brain activity metrics.

In one or more embodiments, the health sensor 130 captures galvanic skin response (GSR) signals. The health sensor 130 may include one or more sensors for measuring electrodermal activity, skin conductance, or other autonomic arousal indicators.

In one or more embodiments, the health sensor 130 captures motion or posture signals. The health sensor 130 may include one or more accelerometers, gyroscopes, or other motion sensors for measuring body position, movement patterns, activity levels, or other kinematic metrics.

In one or more embodiments, the health sensor 130 captures interaction telemetry signals. The health sensor 130 may include one or more sensors for measuring interaction latency, focus consistency, scrolling pauses, typing cadence, hesitation intervals, or other behavioral micro-patterns.

The clinician client device 140 is a computing device operated by a licensed clinician and configured to communicate with the analytics system 110 over the network 160 to receive decision-support artifacts, present clinician-facing interfaces for review and approval of computational outputs, and transmit clinician disposition decisions back to the analytics system 110. The clinician client device 140 may comprise a desktop computer, laptop computer, tablet device, smartphone, or other computing device equipped with a processor, memory, network interface, and display, and may access the analytics system 110 through web-based portals, electronic health record-integrated interfaces, dedicated clinical applications, or application programming interfaces, wherein all communications between the clinician client device 140 and the analytics system 110 are encrypted in transit using Transport Layer Security or equivalent protocols to protect patient health information.

The third-party system 150 provides external data sources, policy repositories, or audit logging services to the analytics system 110. The third-party system 150 may include enterprise knowledge repositories, regulatory compliance databases, external structured data sources, or other systems that provide information or services to support the reasoning infrastructure.

The network 160 provides communication connectivity among the analytics system 110, the patient client device 120, the health sensor 130, the clinician client device 140, and the third-party system 150, enabling bidirectional data exchange for clinical decision support operations. The network 160 may comprise one or more of a local area network, a wide area network, the Internet, a cellular network, a wireless network, a wired network, a virtual private network, or a combination thereof, and may implement communication protocols including Hypertext Transfer Protocol Secure (HTTPS), Health Level Seven (HL7) messaging, Fast Healthcare Interoperability Resources (FHIR) RESTful APIs, WebSocket protocols for real-time telemetry streaming, or other secure communication protocols suitable for healthcare data transmission. All communications transmitted over the network 160 are encrypted in transit using Transport Layer Security (TLS) version 1.2 or later, Advanced Encryption Standard (AES) with key lengths of at least 128 bits, or equivalent cryptographic protocols to protect patient health information during transmission and to ensure compliance with applicable healthcare data privacy regulations.

The architecture supports distributed execution without compromising governance enforcement or traceability. The system maintains deterministic governance enforcement and audit-grade traceability across all deployment configurations, whether centralized or distributed. The modular architecture enables dynamic replacement of individual components, version updates to models or policies, and expansion of framework evaluators without requiring changes to the overall system architecture or compromising the integrity of governance controls.

### System Architecture

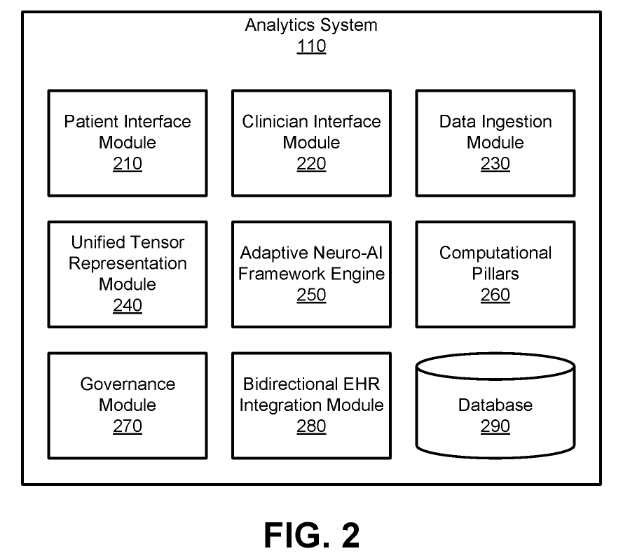

FIG. 2 is a block diagram showing an architecture of the analytics system, according to one or more embodiments. The analytics system 110 includes a patient interface module 210, a clinician interface module 220, a data ingestion module 230, a unified tensor representation module 240, an adaptive neuro-AI framework (ANAF) engine 250, computation pillars 260, a governance module 270, a bidirectional electronic health record (EHR) integration module 280, and a database 290. In other embodiments, the analytics system 110 may include additional, fewer, or different components than those listed herein. In other embodiments, the functionality of the various modules can be disparately distributed across the modules.

The patient interface module 210 maintains a user interface for presentation on the patient client device. The patient interface module 210 renders graphical user interfaces on patient client devices, including text input windows, voice input controls, and multimodal input mechanisms. The patient interface module 210 captures patient-reported outcome measures, behavioral engagement telemetry, and biometric data streams from connected sensors when available and consented.

The patient interface module 210 transmits the captured inputs to downstream processing modules for feature extraction and state vector estimation. The patient interface module 210 also presents decision-support outputs, explanation traces, and uncertainty indicators to the patient through the rendered interface, wherein the outputs are formatted for patient comprehension and include clinician-approved care instructions, monitoring schedules, adherence-support interventions, or some combination thereof.

The patient interface module 210 may generate the patient-facing user interface to guide delivery of pharmacological interventions. In one embodiment, the patient interface module 210 renders interactive medication adherence tracking interfaces that display scheduled medication administration times, dosage instructions, adherence confirmation prompts, or some combination thereof. The module may present medication-related educational content including mechanism-of-action explanations, expected therapeutic timelines, common side-effect profiles, and management strategies for adverse effects, wherein the content is tailored to the patient's current pharmacologic state vector and health literacy level.

The patient interface module 210 may capture patient-reported medication adherence data, including dose-taking confirmation timestamps, missed-dose reports, and reasons for non-adherence, transmitting this telemetry to the adherence-aware pacing controller and the pharmacologic state estimator for incorporation into the closed-loop feedback cycle. In one embodiment, the module implements medication reminder delivery with adaptive timing based on the patient's historical adherence patterns, circadian rhythm data when available, and contextual feasibility constraints including work schedule and daily routine.

The patient interface module 210 may further present side-effect monitoring questionnaires at configurable intervals following medication changes, wherein the questionnaire selection and cadence are determined by the adverse effect sentinel based on the patient's adverse-event susceptibility trajectory. The module formats all medication-related outputs to emphasize that medication decisions require clinician authorization and that the interface serves a decision-support and adherence-facilitation function rather than an autonomous prescribing function.

The clinician interface module 220 maintains a user interface for presentation on the clinician client device. The clinician interface module 220 renders structured decision-support interfaces on clinician-facing devices. The clinician device may support web-based portals, electronic health record (EHR)-integrated in-basket views, mobile clinical applications, or some combination thereof.

The clinician interface module 220 presents results of the computational analyses generated by the computational pillars 260. These results may include ranked diagnostic differentials with posterior probabilities and confidence intervals, risk trajectory forecasts with driver attribution and uncertainty bounds, treatment sequencing proposals with rationale traces identifying constraint satisfactions and optimization trade-offs, medication parameter options with constraint-satisfaction summaries and monitoring plans, operational alerts with priority metadata and escalation recommendations, or some combination thereof.

The clinician interface module 220 captures clinician disposition decisions for each presented output. The decisions may entail approval, modification, deferral, or rejection of actions. The clinician interface module 220 may provide these decisions back to the ANAF engine for further iteration based on the clinician feedback. In embodiments where the clinician approves of the recommended treatment plan, the clinician interface module 220 provide the treatment plan for deployment or execution by the patient interface module 210.

In some embodiments, the clinician interface module 220 may implement acknowledgment confirmation mechanisms for crisis alerts and escalation notifications. This mechanism requests explicit clinician acknowledgment before service-level-agreement timers are satisfied and before escalation state transitions are recorded as complete.

The clinician interface module 220 can further provide, via the user interface, access to attribution traces that identify which input features, data modalities, state-vector dimensions, or constraint evaluations contributed to each computational output. This added insight empowers the clinician to evaluate whether the system's reasoning aligns with clinical judgment and to understand the evidentiary basis for each recommendation. The clinician interface module 220 presents uncertainty annotations that distinguish epistemic uncertainty from aleatoric uncertainty, further empowering the clinician to determine whether additional data collection would resolve ambiguity or whether the ambiguity is intrinsic and must be managed through conservative clinical action.

The clinician interface module 220 further supports bidirectional communication with the EHR integration module 280, enabling retrieval of patient context from the EHR at the point of decision-support artifact review and enabling structured writeback of clinician-approved decisions, assessment results, treatment plan updates, and documentation artifacts to the patient's EHR record. The clinician interface module 220 can execute on the architectural constraint that no computational output constitutes a final clinical action without explicit clinician authorization, such that the system operates as a decision-support tool under clinician governance rather than as an autonomous clinical agent.

The data ingestion module 230 is configured to receive multimodal longitudinal patient data from a plurality of heterogeneous data sources and to perform preliminary processing operations that prepare the data for transformation into the unified temporally indexed tensor representation. The data ingestion module 230 receives data from electronic health record systems via structured application programming interfaces, clinician-entered assessments submitted through clinician-facing interfaces, patient-reported outcome measures captured through mobile applications or web portals, behavioral engagement telemetry generated by digital therapeutic tools, optional biometric sensor streams from wearable devices when available and consented, pharmacy and medication data from pharmacy benefit managers or e-prescribing systems, administrative and operational data from scheduling systems and insurance eligibility services, other third party systems, or some combination thereof.

Upon receipt of each data element, the data ingestion module 230 assigns a cryptographic timestamp and a provenance identifier to enable tamper-evident reconstruction of any prior data state. In one embodiment, the cryptographic timestamp is generated using a hash-chain mechanism in which each new timestamp incorporates the hash of the prior timestamp, creating a tamper-evident sequence that prevents retroactive modification of temporal ordering. The provenance identifier encodes the data source type, source system identifier, extraction method, and any transformations applied during ingestion, enabling complete traceability of each data element from its origin through all downstream processing stages.

The data ingestion module 230 performs preliminary data quality assessment on each incoming data element to identify and flag quality concerns that may affect downstream inference reliability. Quality assessment operations include completeness checking to verify that required fields are populated, range validation to verify that numerical values fall within plausible clinical ranges, temporal consistency checking to verify that timestamps are logically ordered and not duplicated, and format normalization to convert heterogeneous date formats, unit systems, and encoding schemes to canonical representations. Data elements that fail quality checks are flagged with quality-concern indicators that propagate to the tensor representation and condition downstream inference via reduced confidence weights, ensuring that unreliable data contributes less strongly to state-vector updates.

The data ingestion module 230 applies modality-specific preprocessing pipelines to transform raw data from each source into structured feature representations suitable for tensor encoding. For structured rating scales, the module extracts item-level scores and total scores, normalizes them to a common range based on each instrument's maximum possible score, and computes derived features including subscale scores where clinically relevant. For free-text clinician notes, the module applies natural language processing to extract structured features including symptom mentions, severity qualifiers, temporal qualifiers, medication references, and risk-relevant language, with each extracted feature assigned a confidence weight reflecting the extraction model's certainty. For behavioral engagement metadata, the module computes derived features including session attendance rate over configurable windows, homework completion rate, engagement trend indicators, response latency statistics, and pattern regularity metrics. For biometric signals when available, the module applies signal-specific preprocessing including artifact rejection, epoching, and feature extraction to generate summary statistics suitable for clinical interpretation.

The data ingestion module 230 transmits the processed data elements, along with their assigned timestamps, provenance identifiers, quality indicators, and confidence weights, to the unified tensor representation module 240 for incorporation into the temporally indexed tensor structure that serves as the canonical input to the ANAF core engine and all computational pillars.

The unified tensor representation module 240 is configured to transform ingested data into a structured multidimensional tensor T that serves as the canonical input to the ANAF core engine 250 and all five computational pillars. The tensor T may be defined over three axes: a patient axis indexing individual patients, a temporal axis indexing observation times, and a feature axis indexing individual data features. For a given patient p at time t, the tensor entry T[p, t] comprises a feature vector f(p,t) containing modality-specific extracted features normalized to a common scale, a missingness indicator m(p,t) that explicitly represents absent data to prevent imputation artifacts from being treated as observations, a confidence weight vector w(p,t) providing modality-specific reliability estimates that condition downstream inference, a provenance metadata vector s(p,t) linking each feature to its originating data source and extraction method, and a consent-gating flag vector g(p,t) controlling which data elements are available for computation based on patient consent status.

In one embodiment, the tensor T is implemented using a sparse data structure that stores only observed entries, providing computational advantages including incremental update in O(k) time upon receipt of a new observation with k features without needing to perform re-computation over the full temporal history, memory overhead proportional to the number of actual observations rather than the product of patients, time points, and features, streaming inference capability enabling immediate state-vector updates without waiting for batch processing windows, and deterministic reconstruction capability enabling exact reconstruction of any prior tensor state from the sparse observation log for replay purposes. Because clinical data is inherently asynchronous, with different modalities observed at different times and most features unobserved at most time points, the sparse encoding avoids the substantial memory overhead that a dense tensor representation would incur.

The unified tensor representation module 240 applies modality-specific feature extraction pipelines to transform raw data from each source into the standardized feature vector format. For structured rating scales, the module may extract item-level scores and total scores, normalizes them to a common range based on each instrument's maximum possible score, and computes derived features including subscale scores and item-response patterns where clinically relevant. For free-text clinician notes, the module may apply natural language processing to extract structured features including identified symptom mentions, severity qualifiers, temporal qualifiers indicating improvement or worsening, medication references, and risk-relevant language, with each extracted feature assigned a confidence weight reflecting the extraction model's certainty. For behavioral engagement metadata, the module may compute derived features including session attendance rate over configurable windows, homework completion rate, engagement trend indicators, response latency statistics, and pattern regularity metrics. For biometric signals, when available, the module may apply signal-specific preprocessing including artifact rejection, epoching, and feature extraction to generate summary statistics such as sleep onset latency, total sleep time, sleep efficiency, heart-rate variability metrics, circadian rhythm regularity indices, or some combination thereof. The module performs temporal resampling to align features from different modalities to a common temporal grid, retaining actual observation timestamps for intermittently observed features and computing epoch-level summary statistics for continuously sampled signals, wherein the missingness encoding explicitly represents the absence of data between observations rather than interpolating unobserved values.

The adaptive neuro-AI framework (ANAF) engine 250 is the central computational component of the analytics system 110, receiving the unified temporally indexed tensor representation from the unified tensor representation module 240 as input and producing and maintaining five linked probabilistic state vectors that collectively represent the system's estimate of the patient's clinical state across multiple domains. The ANAF engine 250 continuously constructs and updates a Care Delivery State Vector (CDSV) encoding operational and access-related state, a Patient-Specific Temporally Indexed Psychiatric State Vector (PS-TIPSV) encoding diagnostic state, a Care State Vector (CareSV) encoding treatment-related state, a Risk State Vector (RiskSV) encoding destabilization and suicide-risk state, a Pharmacologic State Vector (PharmSV) encoding medication-related state, or some combination thereof, with cross-conditioning between state vectors such that changes in one domain propagate constrained effects to others through defined coupling pathways maintained in a cross-pillar coupling matrix.

Each state vector maintained by the ANAF engine 250 comprises a mean estimate vector representing the best point estimate of the patient's state in that domain, a covariance matrix representing the joint uncertainty across state dimensions, an epistemic uncertainty vector representing the component of uncertainty attributable to insufficient data or model limitation, an aleatoric variance vector representing the component of uncertainty attributable to inherent stochastic variability in the patient's condition, a data sufficiency score vector indicating for each state dimension the quantity and recency of observations supporting the current estimate, and temporal metadata including the timestamp of the most recent update and the number of observations incorporated. The five state vectors are not independent but are coupled through conditional prior updates, wherein when a source state vector changes, affected dimensions in coupled target state vectors have their prior distributions updated to reflect the new conditioning information.

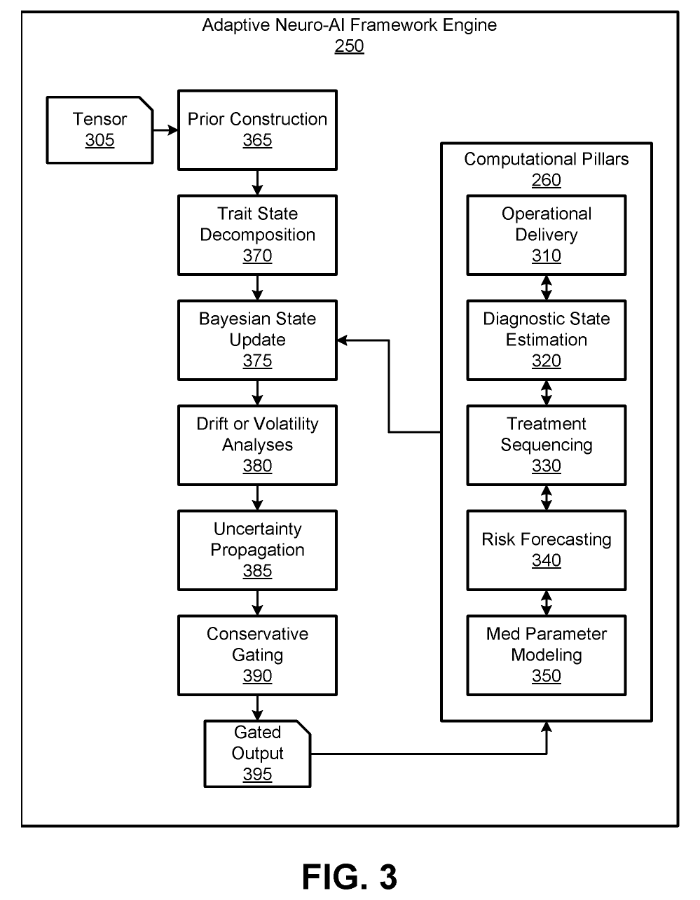

Referring to FIG. 3, FIG. 3 is an illustrative workflow of the ANAF engine, according to one or more embodiments. The ANAF engine 250 executes six computational stages for each state-vector update cycle.

In a first stage, the ANAF engine 250 constructs a prior construction 365 by fitting distributional models to the patient's historical state trajectory. This may entail combining limited patient data with demographically stratified population priors when historical data is insufficient, or by initializing with population-level priors having inflated covariance when no historical data is available, such that initial conservatism is proportional to the system's uncertainty about the specific patient.

In a second stage, the ANAF engine 250 performs trait-state decomposition 370, the engine separates observed patient signals into slowly varying trait components representing enduring patient characteristics and rapidly varying state components representing acute clinical phenomena using hierarchical latent variable models, wherein trait components are updated on longer timescales with higher regularization and state components are updated at each observation with lower regularization, enabling the system to distinguish chronic baseline features from actionable acute changes.

In a third stage, the ANAF engine 250 performs a Bayesian state update 375 via inference upon receipt of new data to produce posterior state estimates incorporating the new observations. The posterior distribution is computed as proportional to the product of the prior distribution and the observation likelihood. The observation likelihood is modulated by confidence weights from the tensor representation such that higher-confidence observations contribute more strongly to the posterior update and features with missingness indicators are excluded from the likelihood computation entirely. In one embodiment, the Bayesian inference is performed using variational inference with parametric posterior approximation, sequential Monte Carlo with weighted particle representation, extended Kalman filtering adapted for psychiatric state spaces, unscented Kalman filtering, or other approximate inference methods suitable for state vectors of the dimensionality required in psychiatric clinical decision support.

In a fourth stage, the ANAF engine 250 computes drift or volatility analyses over the updated state vector history. These analyses may including computing first-order drift representing the rate of change of each state dimension, computing second-order acceleration representing the rate of change of the drift, or computing volatility representing the variance or standard deviation of each dimension over a configurable time window. Moreover, the ANAF engine 250 can compute volatility fingerprints representing patterns of volatility changes across multiple state dimensions that are compared against learned destabilization signatures, and change-point detection identifying statistically significant shifts in the mean, variance, or trend of each trajectory.

In a fifth stage, the ANAF engine 250 performs uncertainty propagation 385, propagating epistemic and aleatoric uncertainty components through all downstream computations using moment propagation for approximately linear computations, sigma-point transforms for moderately nonlinear computations, or Monte Carlo sampling for arbitrary nonlinear computations. This ensures that output uncertainty faithfully reflects input uncertainty and is not artificially narrowed by intermediate processing stages.

In a sixth stage, the ANAF engine 250 performs conservative gating 390 by evaluating propagated uncertainty against configurable thresholds and applies one or more conservative actions when thresholds are exceeded. These conversative actions may include reducing the magnitude of recommended changes, increasing recommended monitoring frequency, narrowing the set of permissible actions to a conservative subset, withholding output pending clinician review, or annotating output with explicit indicators identifying whether elevated uncertainty is primarily epistemic or aleatoric. The gating thresholds are configurable per state-vector dimension, per computational pillar, per clinical context, or some combination thereof, providing adaptability to the system to degrade gracefully under ambiguity by becoming more conservative rather than producing overconfident outputs or withholding output entirely.

The ANAF engine 250 provides the updated state vectors to the computational pillars 260 for generation of decision-support outputs, wherein each pillar consumes one or more state vectors as input and produces clinician-review artifacts including recommended actions, rationale traces, constraint-satisfaction summaries, uncertainty annotations, or some combination thereof. The ANAF engine 250 operates in a continuous closed loop, receiving outcome telemetry from executed workflows through the operational delivery pillar and using the telemetry to trigger state-vector recalibration, comparing observed outcomes against predicted trajectories, updating model confidence, refining individualized priors for subsequent iterations, and logging prediction-versus-outcome comparisons to the model behavior ledger for ongoing calibration monitoring.

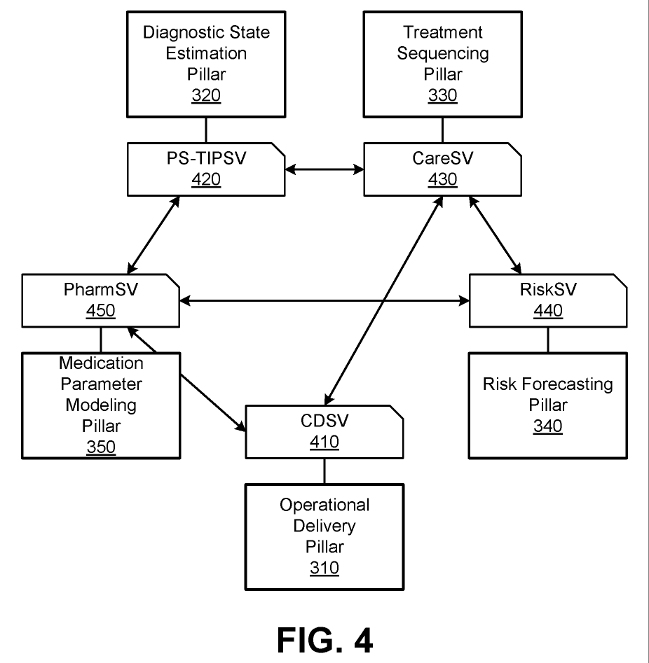

FIG. 4 shows a block diagram of the Operational Delivery Pillar, according to one or more embodiments. The diagram illustrates the cross-pillar coupling matrix that defines directional relationships between the five linked probabilistic state vectors maintained by the ANAF engine 250.

The Patient-Specific Temporally Indexed Psychiatric State Vector (PS-TIPSV) 420 from the diagnostic state estimation pillar conditions the Care State Vector (CareSV) 430 in the treatment sequencing pillar by establishing the diagnostic context within which treatment options are evaluated. The PS-TIPSV 420 further constrains the Pharmacologic State Vector (PharmSV) 450 in the medication parameter modeling pillar by shrinking the permissible medication action space when diagnostic uncertainty is elevated.

The Risk State Vector (RiskSV) 440 from the risk forecasting pillar gates treatment intensity within the CareSV 430 by restricting the permissible intensity and rate of treatment change when destabilization risk is elevated, and further restricts the action space available to the PharmSV 450 by narrowing the set of candidate medication adjustments under elevated risk conditions.

The PharmSV 450 medication volatility indicators feed back to the RiskSV 440 as input features, such that pharmacologic instability is reflected in destabilization forecasts.

The Care Delivery State Vector (CDSV) 410 from the operational delivery pillar bounds what the CareSV 430 and PharmSV 450 may recommend by restricting recommendations to those that are operationally deliverable given current access, capacity, and engagement constraints. The CDSV 410 receives telemetry from executed workflows and feeds observed outcomes back to the ANAF engine to trigger state recalibration across all five state vectors, closing the feedback loop.

The diagram depicts the propagation of uncertainty across pillar boundaries, wherein elevated epistemic uncertainty in one state vector produces corresponding constraint tightening in coupled state vectors, yielding emergent system-wide conservatism without a global conservatism parameter.

Referring back to FIG. 2, the computational pillars 260 provide domain-specific decision-support computations that consume the linked probabilistic state vectors maintained by the ANAF engine 250 and generate clinician-review artifacts for specific clinical domains. Each pillar is implemented as a distinct module executable by one or more processors, receives inputs from and provides outputs to one or more other pillars via the shared ANAF state-vector infrastructure, and may be deployed independently as a standalone system or in combination with one or more other pillars.

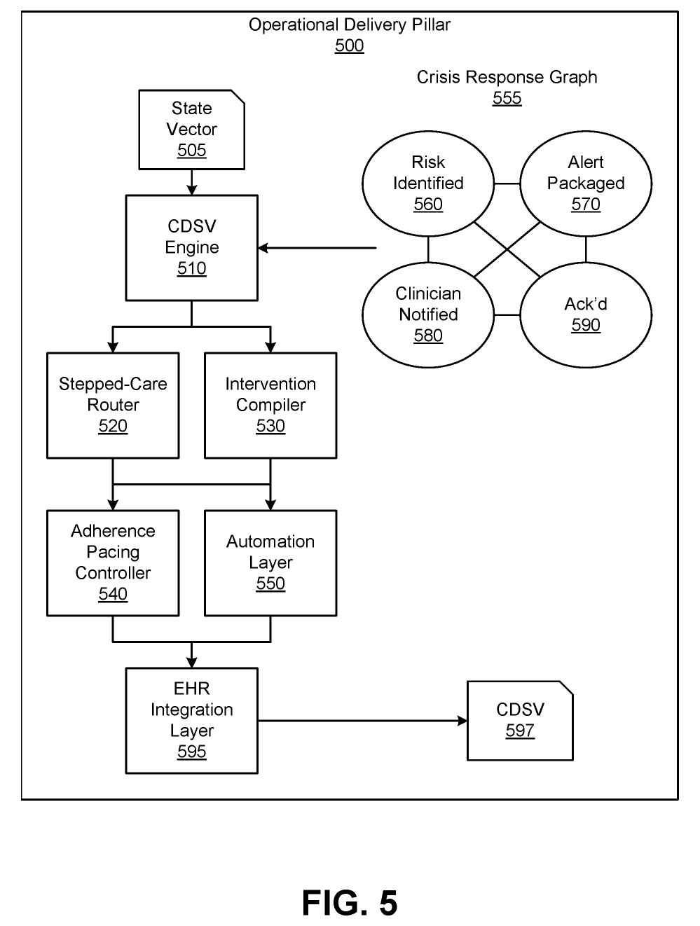

The operational delivery pillar translates clinician-approved computational outputs into executed care by generating workflow-integrated tasks, routing patients across stepped-care tiers, enforcing escalation service-level objectives, automating measurement-based care cadence, and producing structured documentation artifacts for EHR writeback. The diagnostic state estimation pillar constructs the Patient-Specific Temporally Indexed Psychiatric State Vector (PS-TIPSV) using hierarchical probabilistic inference, encoding ranked diagnostic differentials with posterior probabilities, dimensional severity posterior distributions, longitudinal drift features, volatility features, and decomposed uncertainty. The treatment sequencing pillar computes clinician-supervised treatment sequencing proposals using constrained optimization to generate a treatment policy surface bounded by destabilization guardrails and feasibility constraints. The risk forecasting pillar computes multi-horizon hazard trajectories relative to individualized baselines to forecast destabilization risk and suicide-related risk, producing clinician-facing probability distributions with uncertainty bounds and attribution. The medication parameter modeling pillar generates clinician-review medication parameter options within a bounded control framework, wherein suggested actions are constrained by contraindications, interaction rules, destabilization gates, uncertainty conservatism, and governance restrictions, and wherein the system does not autonomously prescribe, modify, or discontinue medications. Details of the various computational pillars are described below in conjunction with FIGS. 5-9.

The governance module 270 validates the output recommended treatment by the ANAF engine 250 and enforces executable condition-action rules within the reasoning pipeline to ensure safety, policy compliance, and domain-of-scope adherence. The governance module 270 receives candidate decision-support outputs and associated reasoning traces from the computational pillars 260 and evaluates them against governance policies and rule nodes comprising hard constraints and soft constraints mapped in a governance rule repository and active policy configurations stored in the database 290. Hard constraints deterministically block, repair, redirect, or escalate disallowed outputs that violate mandatory safety boundaries, regulatory requirements, or domain-of-scope limitations, while soft constraints penalize or repair non-compliant content within the reasoning process prior to final output selection, ensuring governance is embedded within the decision-support pipeline rather than applied as a post-hoc filter.

Operating in parallel with the computational pillars 260, the governance module 270 independently applies executable rules expressed as structured condition-action logic and emits structured compliance indicators specifying which hard constraints were satisfied, which soft constraints were triggered, and what enforcement actions were taken. These compliance indicators feed into output selection alongside framework evaluation scores, conflict metrics, and uncertainty measures computed by the computational pillars, with compliance serving as a mandatory prerequisite for any decision-support output to be presented to the clinician interface module 220. The governance module 270 supports policy-filtered retrieval boundaries that restrict which knowledge elements and treatment options are available for consideration based on jurisdictional overlays, scope-of-practice restrictions, and institutional policy configurations, and implements escalation triggers that route outputs to mandatory clinician review when conflict metrics or uncertainty measures exceed predefined thresholds indicating that automated decision-support may be unreliable.

The governance module 270 contributes to audit-grade traceability by providing rule identifiers specifying which governance rules were evaluated, policy version identifiers enabling deterministic reconstruction of the governance configuration active at the time of each inference, and enforcement action logs documenting which outputs were blocked, repaired, redirected, or escalated and the specific rule violations or constraint triggers that caused each enforcement action. These governance artifacts are included in the structured output packets transmitted to the clinician interface module 220 and in the audit records stored in the version-locked inference log maintained in the database 290, enabling deterministic reconstruction of the complete decision-support process including both the clinical reasoning performed by the computational pillars and the governance enforcement performed by the governance module across centralized or distributed deployment configurations while preserving governance supremacy over personalization preferences derived from longitudinal patient profiles.

The bidirectional EHR integration module 280 provides structured interoperability between the analytics system 110 and external electronic health record systems, enabling the system to retrieve patient clinical data from the EHR and to write back decision-support artifacts, assessment results, and clinician-approved care plans to the patient's EHR record. In one embodiment, the bidirectional EHR integration module 280 supports Fast Healthcare Interoperability Resources (FHIR) R4 or later application programming interfaces for both data retrieval and data writeback, providing standardized structured data exchange that is compatible with a wide range of EHR vendor platforms. The module implements structured writeback capabilities that transmit assessment results including completed patient-reported outcome measures and clinician-entered rating scales to the appropriate sections of the EHR record, clinical decision-support artifacts including diagnostic state summaries, risk trajectory forecasts, and treatment recommendations to the clinician's in-basket or clinical decision-support workspace, treatment plan updates reflecting clinician-approved modifications to care plans to the patient's active treatment plan section, and documentation packs comprising structured clinical summaries, measurement-based care trend reports, and rationale traces formatted for inclusion in progress notes.

The database 290 stores data used by modules of the analytics system 110. For example, the database may store graphs, matrices, tensors, frameworks, policies, etc. In one or more embodiments, the database 290 further stores state vectors, e.g., the five probabilistic state vectors for each patient including the CDSV, PS-TIPSV, CareSV, RiskSV, and PharmSV with their complete temporal histories. The database 290 may further store the unified temporally indexed tensor representation, e.g., with sparse longitudinal encoding of all observed patient data, and the version-locked inference log containing cryptographic timestamps, model version identifiers, hyperparameter states, active constraint sets, input feature snapshots with cryptographic hashes, intermediate computation artifacts, output artifacts, clinician disposition records, or some combination thereof. The database 290 further stores the model behavior ledger comprising an append-only record of calibration statistics, prediction-versus-outcome comparisons, drift indicators, and aggregate performance metrics evaluated on a configurable schedule, governance ledgers recording all constraint evaluations, rule violations, enforcement actions, consent revocation events, and policy configuration changes with timestamps and authenticated user identifiers, longitudinal patient profiles when explicit consent has been granted comprising framework weighting preferences, explanation depth settings, and historical interaction patterns with encryption applied to protect patient privacy, or audit-trace artifacts including complete chains of evidence from raw input through intermediate state vectors and constraint evaluations to final decision-support outputs formatted for regulatory review and post-market surveillance.

### Computational Pillars of the ANAF Engine

FIG. 5 shows a block diagram of the operational delivery pillar 500, according to one or more embodiments. The operational delivery pillar 500 is an embodiment of the operational delivery pillar 310.

The operational delivery pillar 310 comprises a care delivery state vector (CDSV) engine 510 that stores and maintains a time-indexed probabilistic operational state vector encoding access constraints, engagement capacity, care-team topology, transition vulnerability, crisis readiness, service-level-agreement timers, uncertainty bounds with drift estimates, or some combination thereof.

The CDSV Engine 510 receives inputs from administrative data sources, insurance eligibility systems, provider scheduling systems, and behavioral engagement telemetry captured through the patient interface module 210, and processes these inputs to generate posterior distributions over operational state dimensions that condition the feasibility of treatment and medication recommendations generated by other computational pillars. The CDSV maintained by the engine carries uncertainty parameters that are propagated to downstream feasibility assessments, such that recommendations requiring resources with uncertain availability are flagged with appropriately elevated epistemic uncertainty.

The operational delivery pillar 310 comprises a stepped-care router 520 that routes patients among care tiers using constrained directed-graph traversal incorporating risk gates, payer-rule enforcement, capacity constraints, and escalation rules. The care system is modeled as a directed graph in which vertices represent care tiers and edges represent permitted transitions between tiers, with each edge carrying metadata specifying risk gates that define the minimum and maximum RiskSV values for which the transition is permitted, capacity constraints indicating whether the target tier has availability, payer rules specifying whether the transition is covered under the patient's insurance plan and whether prior authorization is required, and escalation rules specifying whether the transition constitutes an escalation to higher-intensity care, a de-escalation to lower-intensity care, or a lateral transfer. In one embodiment, the stepped-care router 520 employs mixed-integer programming to solve a constrained routing problem that identifies the minimum-effective-intensity care tier satisfying all risk gates, capacity constraints, and payer rules, consistent with the stepped-care principle of providing the minimum effective intervention.

The operational delivery pillar 310 comprises an intervention compiler 530 that decomposes clinician-approved care plans into directed acyclic graphs of atomic executable tasks. Each task node in the directed acyclic graph comprises a task type specifying the specific action to be executed, a scheduled execution time or a trigger condition specifying when the task should execute, dependency edges specifying prerequisite tasks that must complete before the current task can execute, a monitoring trigger specifying the conditions under which successful completion is verified and the escalation pathway if completion is not verified within a configurable grace period, and assignment metadata specifying which care-team member or automated system is responsible for execution. The directed acyclic graph structure ensures that tasks are executed in the correct order, that dependencies are satisfied before execution, and that the system can track completion status and trigger downstream tasks or escalations accordingly.

The operational delivery pillar 310 comprises an adherence pacing controller 540 that modulates intervention intensity and cadence based on observed patient engagement to prevent overload and dropout. The adherence pacing controller 540 maintains a dynamic estimate of the patient's current engagement capacity and computes engagement-derived signals including completion confidence representing the probability that the patient will complete the next scheduled intervention, fatigue proxy representing cumulative intervention burden over a configurable time window, disengagement velocity representing the rate of change of engagement metrics over time, and risk-volatility interaction terms that maintain minimum monitoring cadence when the RiskSV indicates elevated risk volatility even if engagement metrics are declining. In one embodiment, the adherence pacing controller 540 implements a proportional-integral control loop in which the controlled variable is the patient's estimated engagement capacity and the manipulated variable is the intervention delivery rate, with a setpoint representing a target engagement level that maximizes therapeutic exposure while maintaining completion confidence above a minimum acceptable threshold.

The operational delivery pillar 310 comprises an automation layer 550 that manages the systematic, repeated administration of standardized assessment instruments to track patient progress and inform state-vector updates. The automation layer 550 performs adaptive cadence scheduling in which the frequency of assessment administration is adjusted based on the patient's current state trajectory, increasing assessment cadence when state-vector drift or volatility is elevated to provide more frequent observations and reduce epistemic uncertainty, and decreasing cadence when the trajectory is stable to minimize patient burden.

The automation layer 550 further performs instrument selection in which the system selects which instruments to administer based on the current diagnostic state and the dimensions with the highest information value, computes response trajectories over time including clinically meaningful change thresholds and treatment-response classification, and generates structured documentation packs summarizing measurement-based care results, trends, and clinical action suggestions formatted for inclusion in clinical notes and EHR writeback. The automation layer 550 may be constrained to operate autonomously within bounds set by the clinician, such that the automation layer 550 is not unbounded in automating care to the patient

The operational delivery pillar 310 comprises an EHR Integration Layer 595 that provides structured interoperability between the analytics system 110 and external electronic health record systems. The EHR Integration Layer 595 supports structured writeback of assessment results, clinical decision-support artifacts, treatment plan updates, and documentation packs to the EHR, in-basket routing of clinician-review artifacts and alert packages to the appropriate provider's EHR in-basket for review and action, questionnaire workflow integration enabling standardized instruments to be administered through the EHR patient portal, appointment and scheduling integration enabling the intervention compiler 530 to create or modify appointments in the EHR scheduling system, and problem list and diagnosis code integration enabling PS-TIPSV diagnostic state information to be referenced against the patient's active problem list.

In one embodiment, the EHR Integration Layer 595 supports Fast Healthcare Interoperability Resources (FHIR) R4 or later application programming interfaces for both data retrieval and data writeback, and further comprises a CDSV 597 that maintains operational state information specific to the EHR integration including connection status, data synchronization timestamps, pending writeback queue status, and error-recovery state.

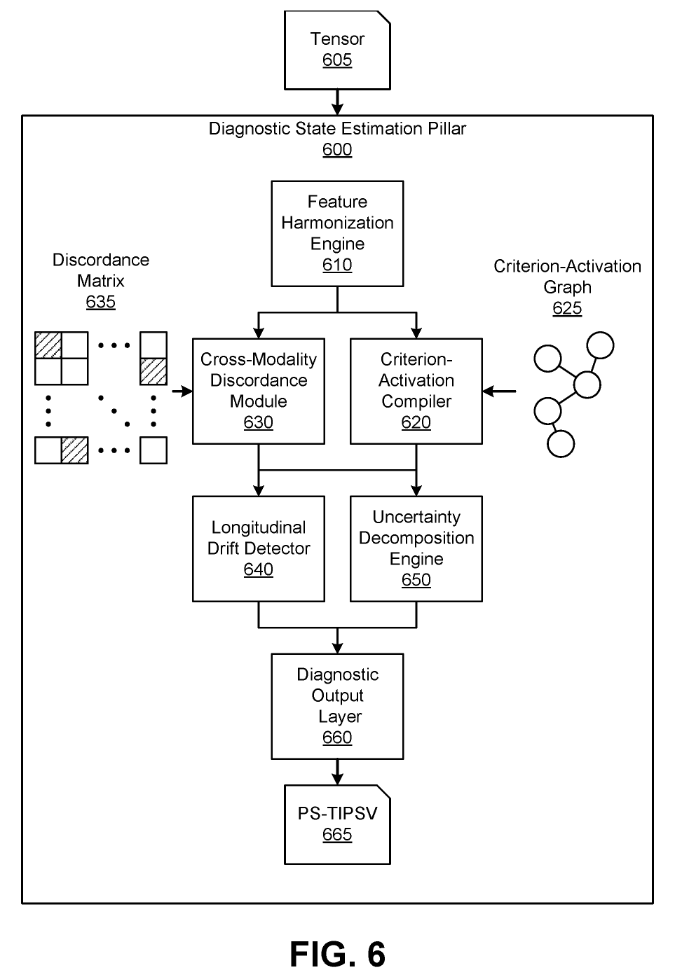

FIG. 6 shows a block diagram of the diagnostic state estimation pillar 600, according to one or more embodiments. The diagnostic state estimation pillar 600 is an embodiment of the diagnostic state estimation pillar 320.

The diagnostic state estimation pillar 320 receives the unified tensor representation 605 from the tensor representation module 240 and processes it through a series of subsystems to construct the Patient-Specific Temporally Indexed Psychiatric State Vector (PS-TIPSV) 665, which encodes the patient's diagnostic state as a continuously updated probabilistic distribution rather than as a categorical classification. The PS-TIPSV 665 represents ranked diagnostic differentials with associated posterior probabilities and confidence intervals, dimensional severity posterior distributions across transdiagnostic clinical constructs, longitudinal drift features comprising first-order velocity and second-order acceleration estimates, volatility features comprising per-dimension variance estimates and multi-dimensional volatility fingerprints, decomposed uncertainty comprising separately tracked epistemic uncertainty parameters and aleatoric variance components, cross-modality discordance indicators identifying statistically significant disagreement among input data sources, and data sufficiency metrics indicating the quantity and recency of observations supporting each state dimension.

The feature harmonization engine 610 receives the tensor representation 600 and performs domain-specific preprocessing to prepare features for diagnostic inference. The harmonization engine applies scale alignment to map different rating instruments measuring overlapping constructs on different scales to a common representational scale using empirically derived crosswalk functions, temporal alignment to annotate each feature with its reference time window enabling the Bayesian state update to correctly associate observations with the time periods they describe, and construct mapping to map features from specific instruments and modalities to a common set of transdiagnostic constructs including depressed mood, anhedonia, sleep disturbance, concentration, psychomotor change, suicidal ideation, anxiety, and irritability. This mapping enables cross-modal comparison and integration such that a sleep-disturbance feature derived from actigraphy can be compared with a sleep-disturbance feature derived from a patient-reported outcome measure.

The criterion-activation compiler 620 encodes psychiatric diagnostic criteria from classification systems into a machine-interpretable probabilistic graphical model represented as a criterion-activation graph 625. Rather than implementing diagnostic criteria as deterministic rule trees in which each criterion is either met or not met, the compiler represents each criterion as a probabilistic node whose activation probability depends on the relevant PS-TIPSV dimensions and their uncertainty. The compiler encodes inclusion criteria, exclusion criteria, temporal requirements, and differential-diagnosis relationships, with exclusion criteria encoded as hard constraints that reduce the posterior probability of a diagnosis to near zero when the exclusion condition is met with high confidence. The system computes a posterior probability distribution over the number of active criteria for each diagnostic hypothesis, yielding a graded confidence in each hypothesis rather than a binary determination.

The cross-modality discordance module 630 evaluates the concordance or discordance between evidence from different data modalities for each transdiagnostic construct. The module computes a discordance matrix 635 in which each entry quantifies the degree of disagreement between two modalities for a specific construct, with the discordance score computed as the variance of the modality-specific estimates normalized by the expected variance under concordance. When a discordance score significantly exceeds unity, indicating that modalities are producing inconsistent evidence, the module inflates the epistemic uncertainty of the PS-TIPSV dimensions associated with the discordant construct, annotates the discordance in the clinician-facing output identifying which modalities disagree and the magnitude of the disagreement, and does not attempt to resolve the discordance by selecting one modality's evidence over another or by averaging the discordant estimates, treating the discordance itself as clinically informative evidence that the clinician should investigate.

The longitudinal drift detector 640 applies the trajectory statistics computed by the ANAF engine 250 specifically to the PS-TIPSV diagnostic dimensions to identify clinically significant trajectory features. The detector identifies pre-threshold deterioration in which a patient's state-component drift is significantly negative even though the absolute level has not yet crossed a diagnostic or severity threshold, accelerating change in which the deterioration rate is itself increasing, volatility destabilization in which state-vector volatility is increasing across multiple diagnostic dimensions simultaneously suggesting broad destabilization, and recovery stall in which an initial improvement trajectory has plateaued with drift approaching zero from the improving direction. These trajectory features enable proactive intervention during the clinical window before threshold-level severity is reached.

The uncertainty decomposition engine 650 applies the general uncertainty decomposition framework to diagnostic inference, producing for each diagnostic dimension and each diagnostic hypothesis an epistemic uncertainty component representing the portion of diagnostic uncertainty attributable to insufficient or conflicting data that could be reduced by additional observation or assessment, an aleatoric uncertainty component representing the portion attributable to genuine clinical ambiguity that cannot be resolved by additional data of the same type, and a data sufficiency indicator quantifying how much observational support the current diagnostic estimate has. The decomposed uncertainty enables the clinician to distinguish between situations in which additional data collection would resolve diagnostic ambiguity and situations in which the ambiguity is intrinsic and must be managed through conservative clinical action.

The diagnostic output layer 660 generates clinician-facing diagnostic outputs that are probabilistic, ranked, attributed, and suitable for regulatory audit. The output layer produces ranked probabilistic differentials comprising a ranked list of diagnostic hypotheses with associated posterior probabilities and confidence intervals rather than a single categorical diagnosis, dimensional severity posterior distributions for each relevant clinical dimension conveying both the system's best estimate and its uncertainty, attribution traces identifying which input features and modalities contributed most to each diagnostic hypothesis enabling the clinician to understand why the system assigned a particular probability to a particular diagnosis, and discordance annotations where the Cross-Modality Discordance Module 630 has identified significant modality disagreement. The Diagnostic Output Layer 660 transmits the PS-TIPSV 665 to other computational pillars for use in treatment sequencing, risk forecasting, and medication parameter modeling, and presents the diagnostic outputs to the clinician interface module 220 for clinician review.

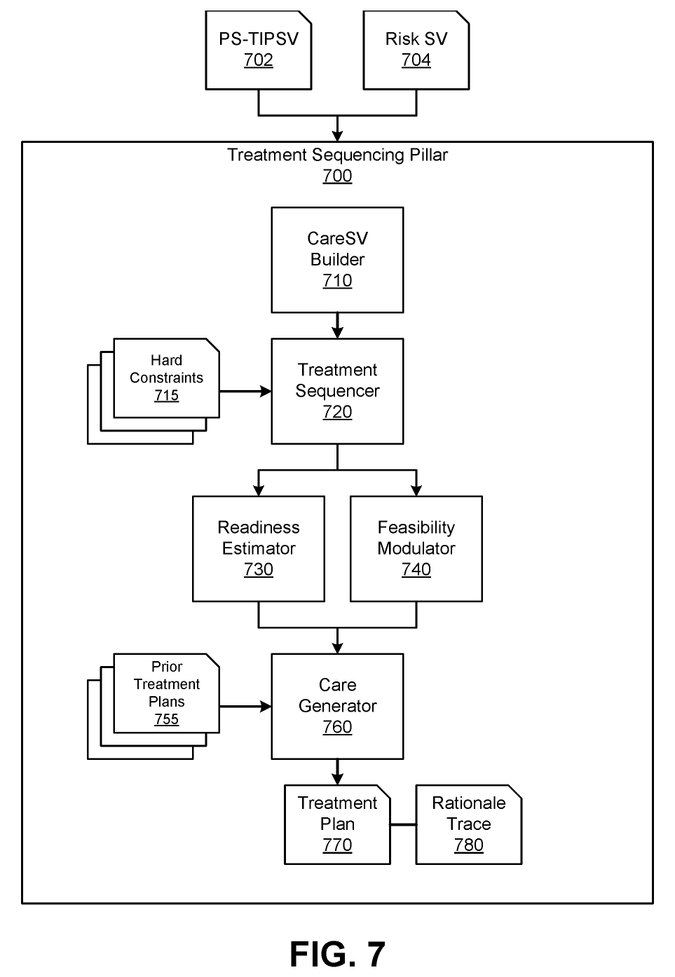

FIG. 7 shows a block diagram of the treatment sequencing pillar 700, according to one or more embodiments. The treatment sequencing pillar 700 is an embodiment of the treatment sequencing pillar 340.

The treatment sequencing pillar 330 receives the PS-TIPSV 702 from the diagnostic state estimation pillar and the RiskSV 704 from the risk forecasting pillar as conditioning inputs, and processes these state vectors through a series of subsystems to compute clinician-supervised treatment sequencing proposals using constrained optimization. The treatment sequencing pillar 330 generates a treatment policy surface that is bounded by destabilization guardrails derived from the RiskSV 704, feasibility constraints derived from the CDSV, cognitive-load budgeting constraints, readiness-window constraints, and scope-of-practice restrictions, wherein the optimization objective balances expected symptom-burden reduction, functional improvement, and treatment engagement maintenance while satisfying all hard constraints.

The CareSV builder 710 constructs a treatment-specific state vector from the PS-TIPSV 702 diagnostic state, the RiskSV 704 risk state, and additional treatment-relevant features extracted from the unified tensor representation. The CareSV builder 710 generates a CareSV that includes a symptom burden profile comprising severity estimates across all relevant symptom dimensions derived from the PS-TIPSV with trait-state decomposition applied, functional status estimates representing impairment across occupational, social, and self-care domains derived from assessment data and behavioral telemetry, engagement probability estimates representing the likelihood that the patient will engage with each available intervention modality derived from the CDSV engagement capacity estimates, resilience proxies representing estimates of the patient's coping capacity, social support availability, and self-efficacy derived from assessment data and historical response patterns, circadian stability estimates representing sleep-wake regularity and circadian rhythm stability when available from actigraphy or sleep-diary data, and contextual feasibility constraints representing real-world factors affecting adherence including schedule availability, transportation access, technology access, and financial constraints derived from administrative data and patient-reported information.

The treatment sequencer 720 is the core optimization engine of the treatment sequencing pillar 330, computing a treatment plan comprising a time-indexed sequence of intervention assignments with intensity and cadence parameters that optimizes an expected-outcome objective function subject to a set of hard and soft constraints. The objective function represents the expected clinical improvement over a planning horizon, formulated in one embodiment as a weighted combination of expected symptom-burden reduction across priority dimensions, expected functional improvement, and expected maintenance of treatment engagement, with weights configurable based on clinician-specified priorities.

Hard constraints define boundaries that the optimizer cannot violate under any circumstances, including a destabilization-risk gate requiring that the predicted destabilization risk under any candidate plan must not exceed a configurable maximum such that when the RiskSV 704 indicates elevated baseline risk the feasible action space is restricted to lower-intensity interventions, a cognitive-load ceiling requiring that the total estimated cognitive burden must not exceed the patient's estimated cognitive-load capacity, scope-of-practice restrictions requiring that proposed interventions fall within the scope of practice of assigned care-team members, consent constraints requiring consistency with the patient's documented consent preferences and treatment agreements, and operational feasibility constraints requiring that the proposed plan be executable given the CDSV operational constraints.

Soft constraints are represented as penalty terms in the objective function and include cognitive-load penalty with increasing penalty as burden approaches the ceiling, schedule-complexity penalty favoring simpler schedules, modality-transition penalty discouraging rapid switching between intervention types, and uncertainty penalty favoring lower-intensity interventions when state-vector uncertainty is elevated.

The readiness estimator 730 identifies timing windows in which the patient is likely to benefit from specific types of interventions, particularly those requiring a minimum level of stability or arousal regulation. The readiness estimator 730 evaluates the patient's current volatility metrics, arousal regulation indicators, and trajectory stability to estimate whether the patient's current state supports demanding interventions such as trauma-focused therapy techniques involving exposure or processing that require the patient to maintain a sufficient window of tolerance. In one embodiment, the readiness estimator 730 identifies neuroplastic windows comprising periods following specific therapeutic modalities in which integration-dependent interventions may be particularly effective, detected by monitoring state-vector dynamics for patterns associated with therapeutic response including a transient increase in volatility followed by stabilization at a new, improved level.

The feasibility modulator 740 adapts treatment recommendations to real-world constraints that materially affect the probability of adherence. The feasibility modulator 740 evaluates schedule constraints to determine whether the patient's work, caregiving, or school schedule accommodates the proposed intervention times, transportation access to determine whether the patient can physically access in-person treatment locations or whether telehealth is a viable alternative, social support to determine whether the patient has support persons who can facilitate treatment engagement through transportation or childcare assistance, technology access to determine whether the patient has the device, connectivity, and digital literacy required for digital interventions, and financial constraints to determine whether the patient's insurance covers the proposed interventions and what the out-of-pocket burden would be. When a proposed intervention has low estimated feasibility, the feasibility modulator 740 substitutes a feasible alternative or adjusts the delivery modality to overcome the identified barrier.

The hard constraints 715 comprise a set of inviolable boundaries that the treatment sequencer 720 cannot violate under any circumstances, stored as structured constraint definitions that are evaluated during the optimization process. Each hard constraint is encoded with a constraint type specifying the category of restriction, a constraint function that evaluates whether a candidate treatment plan satisfies the constraint, a violation severity level indicating the clinical or operational consequences of violating the constraint, and a fallback action specifying what the system should do when no candidate plan satisfies all hard constraints. The hard constraints 715 include destabilization-risk gates that restrict treatment intensification when the RiskSV 704 indicates elevated risk, cognitive-load ceilings that prevent the total estimated cognitive burden from exceeding the patient's capacity, scope-of-practice restrictions that limit interventions to those within the assigned care-team's competencies, consent constraints that enforce the patient's documented treatment preferences, and operational feasibility constraints that restrict recommendations to those executable given current access and capacity limitations.

The care generator 760 receives prior treatment plans 755 from the patient's treatment history and the optimized treatment policy from the treatment sequencer 720, and compiles these inputs into a clinician-reviewable staged treatment plan 770 with an associated rationale trace 780. The staged treatment plan 770 includes a phased treatment timeline with specific interventions assigned to each phase, monitoring triggers specifying the conditions under which the plan should be re-evaluated including symptom burden increases exceeding a configurable threshold, engagement drops below a threshold, or change-point detection, escalation conditions specifying when the plan should be escalated to a higher-intensity tier, and de-escalation conditions specifying when the patient may step down to a lower-intensity tier. The rationale trace 780 provides for each intervention assignment a citation to the optimization objective, the binding constraints that shaped the recommendation, and the trade-offs made when multiple objectives or constraints were in tension, enabling the clinician to understand the computational reasoning underlying each recommendation and to evaluate whether the system's reasoning aligns with clinical judgment.

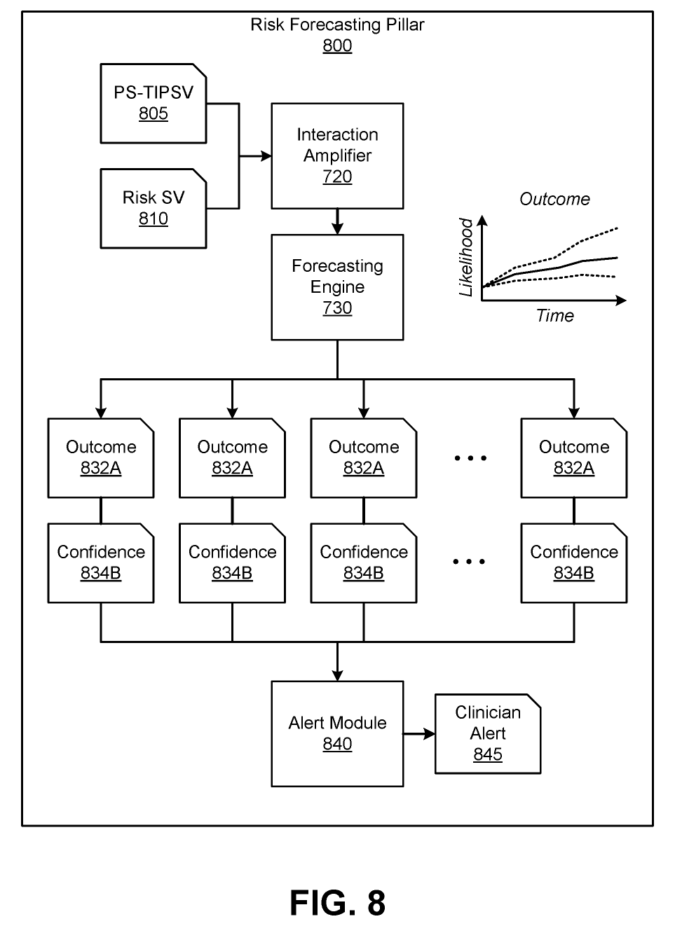

FIG. 8 shows a block diagram of the risk forecasting pillar 800, according to one or more embodiments. The risk forecasting pillar 800 is an embodiment of the risk forecasting pillar 340.

The risk forecasting pillar 340 receives the PS-TIPSV 805 from the diagnostic state estimation pillar and the RiskSV 810 from its own prior update cycle as inputs, and processes these state vectors through a series of subsystems to compute multi-horizon hazard trajectories relative to individualized baselines. The risk forecasting pillar 340 generates probabilistic risk forecasts across short-term horizons spanning hours to days suitable for acute safety planning and crisis prevention, and medium-term horizons spanning weeks to months suitable for treatment planning and care-tier decisions, wherein each forecast comprises a probability distribution over future risk states at each time point in the horizon rather than a single point prediction, conveying both the expected trajectory and the system's uncertainty about the trajectory.

The interaction amplifier 820 models cross-signal interaction effects that elevate risk beyond what additive scoring would predict, addressing the clinical observation that certain combinations of risk factors interact synergistically. The interaction amplifier 820 maintains a library of learned interaction patterns, each specifying a combination of state-vector dimensions whose joint elevation produces nonlinear risk amplification, characterized by the participating dimensions, the activation threshold for each dimension representing the level at which the dimension contributes to the interaction, and the amplification function specifying how the joint presence of the participating dimensions amplifies the risk estimate beyond the additive contribution. The interaction amplifier 820 evaluates the current RiskSV 810 and PS-TIPSV 805 against all interaction patterns and, when one or more patterns are activated, applies the corresponding amplification to the risk forecast, with activated interaction patterns included in the attribution traces provided to clinicians to explain which specific factor combinations are driving the elevated risk estimate.

The forecasting engine 830 generates the multi-horizon probabilistic risk trajectories by projecting the current RiskSV 810 posterior distribution forward in time through a learned state-transition model that incorporates the patient's drift, acceleration, and volatility dynamics computed by the ANAF engine 250. In one embodiment, the forecasting engine 830 uses stochastic state-space projection in which the state-transition model accounts for the patient's planned interventions from the treatment sequencing pillar and current medication regimen from the medication parameter modeling pillar as conditioning variables, such that the forecast reflects the expected effect of the current treatment plan rather than assuming no intervention. The forecasting engine 830 produces for each time horizon a probability distribution over risk states with calibrated confidence bounds that widen monotonically as the forecasting horizon increases, reflecting the increasing uncertainty inherent in longer-term predictions.

The forecasting engine 830 outputs a plurality of outcome predictions 832A, each associated with a confidence estimate 834B that quantifies the reliability of the prediction based on the propagated epistemic and aleatoric uncertainty from the input state vectors. The outcome predictions 832A and confidence estimates 834B are transmitted to an alert module 840 that evaluates whether any predicted outcome exceeds a configurable risk threshold warranting clinician notification.

When a threshold exceedance is detected, the alert module 840 applies alert fatigue suppression logic including temporal clustering to combine multiple alerts generated within a short time window into a single composite alert, cooldown logic to suppress subsequent alerts for the same risk dimension within a configurable period unless the risk estimate increases significantly beyond the level that triggered the original alert, and confidence gating to suppress alerts when the risk estimate carries elevated epistemic uncertainty below a configurable confidence threshold. When an alert passes the suppression filters, the alert module 840 packages the alert with priority metadata reflecting the urgency, confidence, and clinical significance, driver attribution identifying which risk factors and state-vector dimensions are contributing most to the risk estimate, and explainability annotations describing the key factors in plain language suitable for clinical documentation, and transmits the packaged alert as a clinician alert 845 to the clinician interface module 220 for presentation and acknowledgment tracking.

The risk forecasting pillar 340 transmits the updated RiskSV to other computational pillars where it serves as a gating input that restricts the feasible action spaces in the treatment sequencing pillar and the medication parameter modeling pillar. When the RiskSV indicates elevated destabilization risk, the treatment sequencer's feasible action space is restricted to lower-intensity interventions, and the medication parameter optimizer's feasible action space is restricted to exclude medication changes predicted to increase volatility, implementing the cross-pillar constraint propagation architecture in which risk state constrains treatment and pharmacologic decision-making to ensure that safety considerations dominate performance optimization objectives.

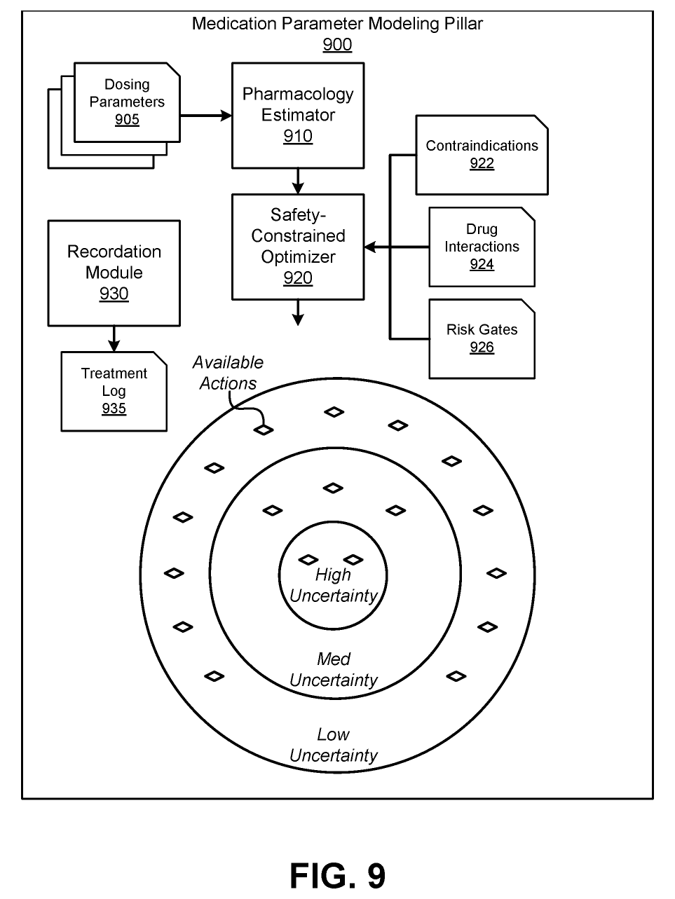

FIG. 9 shows a block diagram of the medication parameter modeling pillar 900, according to one or more embodiments. The medication parameter modeling pillar 900 is an embodiment of the medication parameter modeling pillar 350.

The medication parameter modeling pillar 900 receives the PS-TIPSV from the diagnostic state estimation pillar, the RiskSV from the risk forecasting pillar, and the CareSV from the treatment sequencing pillar as conditioning inputs, and processes these state vectors through a series of subsystems to generate clinician-review medication parameter options within a bounded control framework. The medication parameter modeling pillar 350 operates under the architectural constraint that the system does not autonomously prescribe, modify, or discontinue any medication, with all outputs presented as clinician-review options accompanied by rationale traces, constraint-satisfaction summaries, expected-effect profiles with uncertainty bounds, monitoring plans, and reversibility assessments, wherein the clinician retains sole authority to accept, modify, or reject any suggested medication parameter.

The pharmacology estimator 910 models the patient's inferred pharmacologic state as the Pharmacologic State Vector (PharmSV), a time-varying probabilistic estimate that encodes the dynamic interaction between the individual patient and their medication regimen rather than static drug properties. The PharmSV maintained by the pharmacology estimator 910 includes a stability-flexibility balance representing the degree to which the current medication regimen is stabilizing the patient's condition by reducing volatility versus maintaining flexibility by preserving the patient's capacity for adaptive therapeutic change, an adverse-event susceptibility trajectory representing a time-varying estimate of the patient's susceptibility to adverse effects informed by medication history, reported side effects, dose-response patterns, metabolic indicators where available, tolerance dynamics representing estimates of pharmacologic tolerance development derived from longitudinal dose-response analysis, metabolic indicators from pharmacogenomic testing, laboratory metabolic panels, or drug-level assays when available, associated uncertainty covariance with epistemic and aleatoric decomposition, or some combination thereof. The pharmacology estimator 910 updates the PharmSV using Bayesian inference conditioned on observed medication adherence data from pharmacy records and patient-reported telemetry, observed symptom trajectory changes temporally associated with medication adjustments, reported adverse effects captured through side-effect monitoring questionnaires, and laboratory or pharmacogenomic data when available.

The safety-constrained optimizer 920 selects from a medication control-action library comprising parameterized medication actions including dose increases, dose decreases, titration initiations, taper initiations, timing changes, formulation changes, medication additions, and medication discontinuations, each encoded with hard constraint metadata specifying contraindication lists, drug-drug interaction lists, maximum dose ceilings, minimum dose floors, and titration-rate limits, and soft constraint metadata specifying preferred dose increments, typical titration schedules, and monitoring requirements. The safety-constrained optimizer 920 applies constrained optimization subject to hard constraints that are inviolable, including contraindication matrices that exclude any medication change creating a contraindicated state, drug-drug interaction graphs that exclude medication changes creating clinically significant interactions with major interactions treated as hard constraints and moderate interactions treated as soft constraints with penalty terms, and destabilization risk gates that exclude medication actions predicted to increase volatility when the RiskSV indicates elevated destabilization risk.

The safety-constrained optimizer 920 further applies uncertainty-driven action-space shrinking in which the optimizer defines an uncertainty-adjusted action radius that is a monotonically decreasing function of the epistemic uncertainty from the PS-TIPSV diagnostic state and the PharmSV pharmacologic state, such that as epistemic uncertainty increases the action radius decreases and only actions whose expected impact magnitude is within the radius are considered feasible. Under maximal uncertainty, the action radius may shrink to zero such that only maintenance of the current medication regimen and increased monitoring actions remain feasible, ensuring that the system never recommends aggressive medication changes when it lacks confidence in the underlying diagnostic or pharmacologic state estimates. The optimizer incorporates contraindications 922, drug interactions 924, and risk gates 926 as hard constraints that define the boundaries of the feasible action space before uncertainty-driven shrinking is applied.

The recordation module 930 maintains a treatment log 935 that records all medication parameter options generated by the safety-constrained optimizer 920, the clinician's disposition of each option including approval, modification, deferral, or rejection with timestamps, the rationale traces and constraint-satisfaction summaries associated with each option, and the observed outcomes following clinician-approved medication changes including symptom trajectory changes, adverse-effect reports, adherence patterns, and laboratory results when available. The treatment log 935 provides the historical medication data that informs the pharmacology estimator's 910 updates to the PharmSV, enables the adverse effect sentinel to detect emergent side-effect patterns by comparing pre-change and post-change state-vector trajectories, and supports the deterministic replay capability by preserving the complete medication decision context including which options were considered, which constraints were active, and what uncertainty levels were present at the time of each decision.

The medication parameter modeling pillar 900 implements a learning-window protection mechanism that restricts medication adjustments during periods when the patient is engaged in high-sensitivity therapeutic processes identified by the treatment sequencing pillar, wherein certain therapeutic modalities including trauma-focused therapies, exposure-based therapies, and integration-dependent therapeutic processes require pharmacologic stability to achieve their therapeutic effect. When a protection window is active, the safety-constrained optimizer 920 restricts the set of permissible medication actions to those that do not alter the patient's neurochemical state in ways that would disrupt the ongoing therapeutic process, permitting maintenance doses while restricting dose changes, additions, and discontinuations unless the RiskSV trajectory indicates that the restriction would itself pose an unacceptable safety risk, in which case the conflict is escalated for clinician review with explicit annotation of the competing constraints.

The medication parameter modeling pillar 900 further implements adverse-effect surveillance through continuous monitoring of state-vector dimensions known to be associated with common adverse effects including sleep disruption, weight change, psychomotor change, sexual dysfunction, cognitive effects, or some combination thereof. When a statistically significant change is detected in one or more of these dimensions temporally proximate to a medication change, the pillar generates an adverse-effect hypothesis with a probability estimate and triggers increased monitoring cadence for the affected dimensions, a clinician alert identifying the temporal relationship between the medication change and the observed state-vector shift, and an annotation in the PharmSV updating the patient's adverse-event susceptibility trajectory to reflect the observed pattern. The medication parameter modeling pillar 900 transmits the updated PharmSV to the risk forecasting pillar where medication volatility indicators feedback as input features to the RiskSV estimation, implementing the cross-pillar coupling pathway in which pharmacologic instability is reflected in destabilization forecasts.

### Example Flowchart

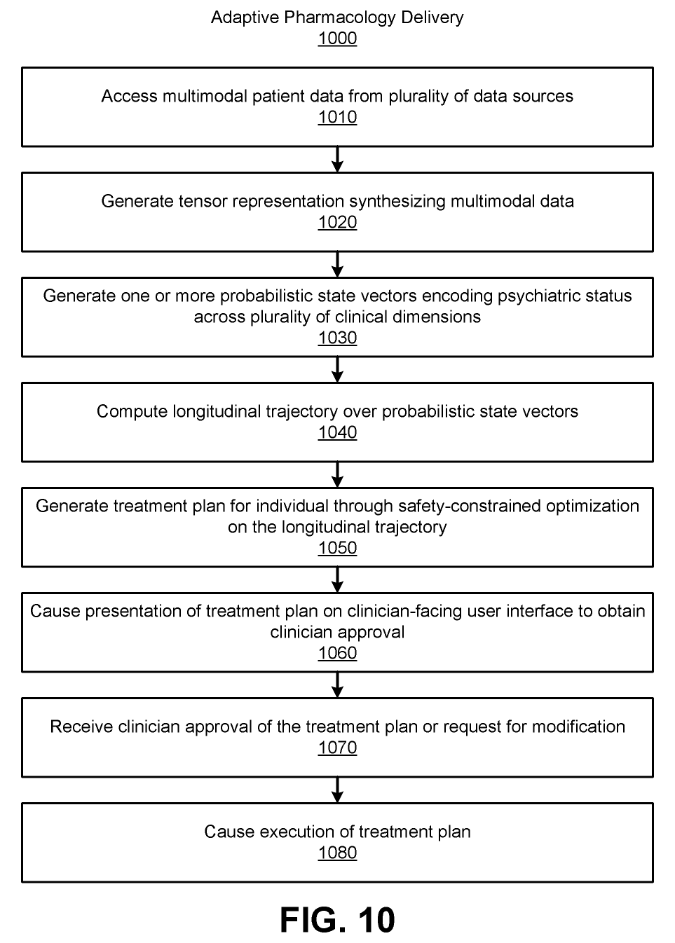

FIG. 10 is a flowchart of adaptive pharmacological treatment delivery, according to one or more embodiments.

The analytics system 110 accesses 1010 multimodal patient data from a plurality of data sources including electronic health records, patient-reported outcome measures, behavioral engagement telemetry, biometric sensor streams when available and consented, pharmacy and medication data, and administrative data. The data ingestion module 230 receives the multimodal data and assigns cryptographic timestamps and provenance identifiers to each data element to enable tamper-evident reconstruction of any prior data state. The data ingestion module 230 performs preliminary data quality assessment including completeness checking, range validation, temporal consistency checking, and format normalization, flagging data elements that fail quality checks with quality-concern indicators that propagate to downstream processing stages.

The analytics system 110 generates 1020 a tensor representation synthesizing the multimodal data into a unified temporally indexed structure comprising feature vectors, missingness encodings, confidence weights, source provenance metadata, and consent-gating flags. The unified tensor representation module 240 applies modality-specific feature extraction pipelines to transform raw data from each source into standardized feature representations, normalizing scales to a common range, aligning temporal references, and mapping features to a common set of transdiagnostic clinical constructs. In one embodiment, the tensor is implemented using sparse longitudinal encoding that stores only observed entries, enabling incremental updates upon receipt of new data without full re-computation and deterministic reconstruction of any prior tensor state from logged observations.

The analytics system 110 generates 1030 one or more probabilistic state vectors encoding psychiatric status across a plurality of clinical dimensions including diagnostic state, care state, risk state, pharmacologic state, and operational delivery state. The ANAF engine 250 performs individualized prior construction by fitting distributional models to the patient's historical state trajectory when sufficient data is available, combining limited patient data with demographically stratified population priors when historical data is insufficient, or initializing with population-level priors having inflated covariance when no historical data is available such that initial conservatism is proportional to the system's uncertainty about the specific patient. The ANAF engine 250 applies trait-state decomposition to separate observed patient signals into slowly varying trait components updated on longer timescales with higher regularization and rapidly varying state components updated at each observation with lower regularization, enabling the system to distinguish chronic baseline features from actionable acute changes.

The analytics system 110 computes 1040 a longitudinal trajectory over the probabilistic state vectors by calculating first-order drift representing the rate of change of each state dimension, second-order acceleration representing the rate of change of the drift, and volatility representing the variance of each dimension over a configurable time window. The ANAF engine 250 computes volatility fingerprints representing patterns of volatility changes across multiple state dimensions that are compared against learned destabilization signatures to identify trajectory dynamics that match patterns historically associated with clinical deterioration. The ANAF engine 250 applies change-point detection using sequential statistical tests to identify statistically significant shifts in the mean, variance, or trend of each trajectory, flagging dimensions that have undergone abrupt changes that might be missed by smoothed drift and volatility estimates.

The analytics system 110 generates 1050 a treatment plan for the individual through safety-constrained optimization on the longitudinal trajectory, wherein the optimization is subject to hard constraints including destabilization-risk gates, cognitive-load ceilings, scope-of-practice restrictions, consent constraints, and operational feasibility constraints. The treatment sequencer 720 optimizes an expected-outcome objective function representing expected clinical improvement over a planning horizon, formulated as a weighted combination of expected symptom-burden reduction, expected functional improvement, and expected maintenance of treatment engagement. The safety-constrained optimizer 920 in the medication parameter modeling pillar applies uncertainty-driven action-space shrinking that defines an uncertainty-adjusted action radius as a monotonically decreasing function of epistemic uncertainty, progressively restricting the set of candidate medication actions as diagnostic or pharmacologic uncertainty increases such that under maximal uncertainty only maintenance and increased-monitoring actions remain feasible.

The analytics system 110 causes 1060 presentation of the treatment plan on a clinician-facing user interface to obtain clinician approval. The clinician interface module 220 may generate the interface presenting the plan as a structured decision-support artifact comprising recommended actions, rationale traces identifying the state-vector dimensions and data sources contributing to each recommendation, constraint-satisfaction summaries documenting which safety constraints were evaluated and satisfied, and uncertainty annotations distinguishing epistemic uncertainty from aleatoric uncertainty. The clinician interface module 220 renders the artifact in the clinician's EHR in-basket or clinical decision-support workspace, providing access to attribution traces that enable the clinician to evaluate whether the system's reasoning aligns with clinical judgment. The system operates under the architectural constraint that no computational output constitutes a final clinical action without explicit clinician authorization, such that no diagnosis is rendered, no medication is prescribed or modified, and no crisis-escalation protocol is activated without clinician approval.

The analytics system 110 receives 1070 clinician approval of the treatment plan or a request for modification, capturing the clinician's disposition decision through the clinician interface module 220 as an approval, modification, deferral, or rejection with timestamp and authenticated clinician identifier. When the clinician approves the treatment plan, the intervention compiler 530 decomposes the approved plan into a directed acyclic graph of atomic executable tasks with dependencies, monitoring triggers, and escalation conditions, and the adherence pacing controller 540 modulates intervention delivery intensity based on completion confidence, fatigue proxies, and disengagement velocity to prevent patient overload. When the clinician requests modification, the system incorporates the clinician's modifications into the treatment plan and re-evaluates constraint satisfaction and uncertainty bounds before proceeding to execution.

The analytics system 110 executes 1080 the treatment plan by transmitting workflow-integrated tasks to the patient interface module 210 for presentation to the patient, scheduling standardized assessment instruments at adaptive cadence determined by state-vector drift and volatility metrics, and capturing outcome telemetry from executed workflows including assessment completion status, engagement signals, adherence patterns, and subsequent clinical measurements. The operational delivery pillar compiles clinician-approved outputs into operationally executable workflow artifacts with task schedules, monitoring triggers, stepped-care routing instructions, and adherence-aware pacing parameters that are transmitted to the patient client device 120 and integrated with the patient's EHR record through the bidirectional EHR integration module 280. The captured outcome telemetry is fed back to the ANAF engine 250 to trigger state-vector recalibration in a closed loop, wherein the system compares observed outcomes against predicted trajectories, updates model confidence, refines individualized priors for subsequent iterations, and logs prediction-versus-outcome comparisons to the model behavior ledger for ongoing calibration monitoring.

### Example Machine-Learning Models

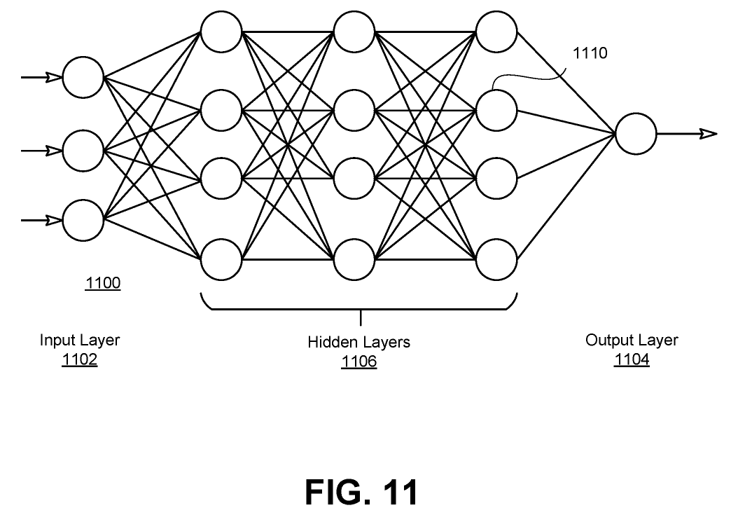

FIG. 11 shows a block diagram of a neural network model, according to one or more embodiments. The neural network 1100 may receive an input and generate an output. The input may be the multimodal feature vector derived from patient data (text, audio, video, physiological signals), and the output may be predictions of current state variables or proposals for therapeutic interventions. The network may include convolutional layers for processing visual data, transformer layers for text semantics, and recurrent sequences for temporal correlation across sessions.

The order and number of layers may vary by modality. Convolutional layers may be used for facial-expression detection; recurrent or transformer layers may model conversation dynamics and affective trajectories. Kernel sizes and attention heads may differ for processing fine-grained emotional cues versus longer temporal dependencies.

Training may include forward propagation and back-propagation across nodes associated with functions such as convolution, pooling, attention weighting, and activation (e.g., ReLU, tanh). Each node’s operation reflects transformations relevant to emotion recognition, language understanding, or physiological signal interpretation.

Training of a machine learning model may include iterative forward and backward passes using mental-health session data. For instance, a computing device may receive a training set of past multimodal sessions labeled with therapeutic outcomes. For each training sample, predicted emotional state or therapy effectiveness is generated and compared with clinician-verified labels. The system adjusts network weights through stochastic gradient descent to minimize the chosen loss function.

Each function in the neural network may include coefficients adjusted during training. Activation functions (ReLU, sigmoid, tanh) control nonlinear mapping of extracted features representing voice prosody or text sentiment. Performance is evaluated by comparing predictions (e.g., mood state change, engagement score) to ground-truth outcomes measured post-therapy.

Multiple training rounds may be performed until convergence, after which the trained model infers patient states or generates interventions during live sessions. The trained model predicts risk, engagement decay, or therapeutic response probability for decision support in ongoing care.

In some embodiments, the system periodically retrains the model on newly collected session data to improve accuracy and adapt to patient population drift. Retraining may occur as part of a continuous-learning cycle in which each verified intervention outcome updates the training corpus and fine-tunes the generative and inference models for better personalization and safety alignment.

In some embodiments, model distillation may transfer knowledge from large generative or multimodal reasoning models to smaller local models within the analytics system 140. For instance, a remote transformer-based generative model (teacher) may generate therapeutic recommendations, and a simplified local model (student) may learn from those outputs to operate on edge devices with reduced latency and footprint.

Feature-based distillation may align embeddings between the teacher model’s multimodal transformer and a student model, preserving latent affective and linguistic features while reducing computational cost. Hybrid approaches may combine response- and feature-level distillation to maintain therapeutic interpretability and efficiency.

This distillation process allows clinical AI deployments (e.g., on patient mobile apps or clinician dashboards) to achieve inference consistency while meeting regulatory and safety constraints, ensuring lower latency, privacy protection, and compliant operation in healthcare contexts.

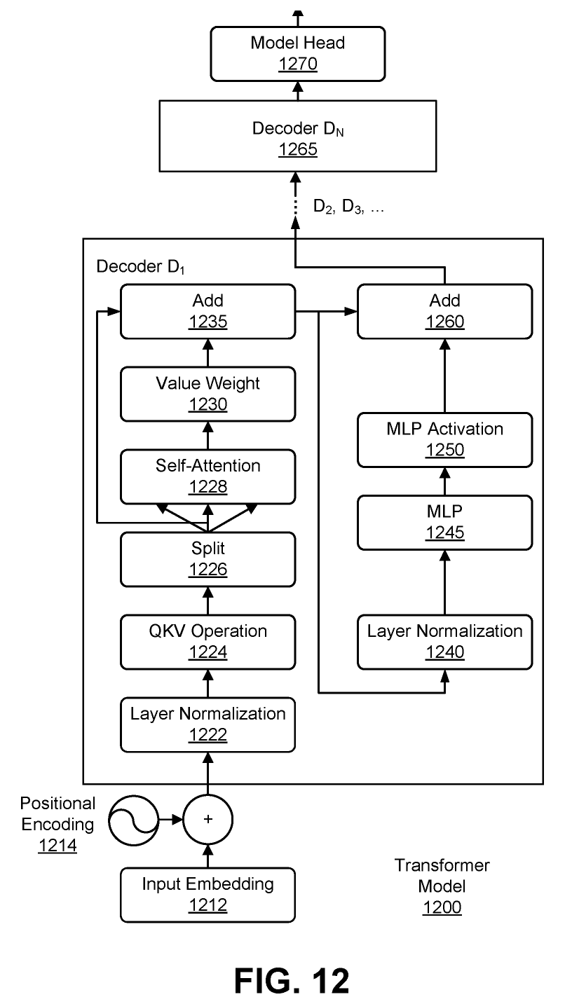

FIG. 12 is a conceptual diagram of functional blocks of a transformer-based neural network model 1200, in accordance with some embodiments. For simplicity, the transformer-based neural network model 1200 is referred to as a transformer model 1200. The transformer model is an example of a machine-learning model discussed in this disclosure. An actual transformer model 1210 may be a large language model that involves numerous neurons, such as a large number of decoders and parameters. The structure illustrated in FIG. 12 is part of a decoder for generating token attention. In a language-processing task related to therapeutic reasoning and intervention generation, the input may take the form of a sequence of words representing a structured prompt encoding multimodal state features and graph context. Each token represents a respective embedding in a latent space. Based on the input tokens, the transformer model 1200 repeatedly generates a sequence of output tokens in an autoregressive manner that correspond to candidate therapeutic actions or interpretive rationales for clinical prompts.

In some embodiments, a transformer model 1200 includes a set of N decoders, D1, D2, … DN. Each decoder receives input representations and generates output representations. For example, the first decoder D1 generates intermediate embeddings contextualized for patient psychological state variables and multimodal cues. Each subsequent decoder refines these embeddings using prior decoder outputs and the state-graph context until a final therapeutic recommendation vector is produced. Some decoders may correspond to analyzing data dimensions that model text, audio, video, and physiological indicators. These multimodal streams are used to perform feature integration and therapeutic inference, enabling the model to reason across linguistic, affective, and behavioral data.

The transformer model 1200 may include a model head block 12120 that receives the set of output representations from the final decoder DN and generates an output token as the output for the current iteration. This output may represent natural-language therapeutic guidance, clinician-facing documentation text, or intervention rationale according to safety and regulatory policies managed by the analytics system.

As shown in FIG. 12, a decoder in the transformer model 1200 includes a first layer-normalization block 1222, a query-key-value (QKV) operation block 1224, a split block 1226, a self-attention block 1228, a value-weight block 1230, a first add block 1235, a second layer-normalization block 1240, multi-layer perceptron (MLP) block 1245, an MLP activation block 1250, and a second add block 1260. The operations in the first decoder D1 are exemplary; subsequent decoders may include similar operations. These layers allow the model to attend dynamically to relevant features in multimodal inputs and to latent state-graph variables describing the patient’s historical therapeutic context.

FIG. 12 illustrates a flow for the attention mechanism of a transformer model 1200. The transformer model 1200 receives an input sequence such as encoded state-graph data and multimodal embeddings collected from patient and clinician-facing interfaces. Each symbol is converted into a token that takes the form of an embedding vector. The sequence of symbols is represented as a matrix of embedding vectors, each embedding arranged in a row of the matrix. The layer-normalization block 1222 receives the matrix and normalizes its values to stabilize input variance across sessions and modalities.

During training, the transformer model 1200 may be trained in an autoregressive manner using masked label prediction. The input may be a therapeutic prompt sequence encoding prior session data and partially masked outcome labels. To simulate intervention prediction, the system applies masking where unknown intervention types or outcomes are hidden. The decoder attends only to previously observed state nodes and validated interventions while predicting masked positions corresponding to outcome nodes. The objective minimizes prediction error between masked positions and true therapeutic identifiers, enabling the transformer to model long-range dependencies and infer causal relationships between patient states and therapy outcomes.

The QKV operation block 1224 receives the normalized dataset and performs projections to generate query, key, and value matrices. The operation applies learned weights to align representations with contextual signals derived from the longitudinal clinical memory graph (LCMG). The QKV operation models relationships among psychological variables, intervention history, and multimodal affect markers to produce attention distributions guiding therapeutic reasoning and content generation.

The split block 1226 splits the QKV output into query, key, and value matrices. The self-attention block 1228 uses these matrices to generate an attention matrix, applying softmax scaling. The softmax converts logit scores into attention probabilities indicating relevance between patient states and proposed interventions. This attention function allows the transformer model 1200 to associate multimodal patterns with therapy outcomes and prioritize nodes with higher causal relevance within the longitudinal graph.

The value-weight block 1230 receives the attention-score data to generate an attention dataset representing weighted combinations of value vectors. The results are concatenated in the add block 1235 and further normalized by 1240. These operations refine the interpretive context and produce latent embeddings that encode therapeutic rationale, patient progress indicators, and confidence metrics for graph updates.

Each decoder may include one or more MLP blocks and 3and MLP activation blocks 1250 configured with nonlinear activation functions. The activation functions introduce non-linearity and support mapping of complex psychological transitions. Common activations may include ReLU, tanh, sigmoid, or GeLU. The MLP layers perform feature extraction across multimodal signals, generate compact context embeddings, and select token sequences for subsequent decoding related to therapy planning. Outputs are concatenated by and 3to complete the cross-modal fusion pipeline.

The output of the first decoder D1 is passed to subsequent decoders until final output data are generated. Each decoder may operate with different trained parameters focused on progressively refined aspects of patient mental-state modeling or intervention inference. The model head block 12120 receives the final output from DN and determines an output token that forms natural-language recommendations or log entries. A softmax operation performed at the LM head selects the next token, resulting in governed therapeutic language or clinician summary text integrated into longitudinal documentation of patient progress.

### Working Example

The following illustrative example demonstrates the operation of the ANAF engine and computational pillars using synthetic patient data. All values are illustrative and do not represent actual patient information.

Synthetic Patient Profile. Consider a patient, referred to herein as Patient A, presenting with the following initial clinical data at time t=0: a Patient Health Questionnaire-9 (PHQ-9) score of 14 (moderate depression range), a Generalized Anxiety Disorder-7 (GAD-7) score of 11 (moderate anxiety), a current medication regimen of sertraline 100 mg daily initiated approximately 8 weeks prior, three completed therapy sessions over the preceding 6 weeks, and sleep data from a wrist-worn actigraphy device indicating 72% sleep efficiency with a sleep onset latency of 38 minutes. Patient A has 8 prior clinical observations spanning 14 weeks.

Tensor Construction. The data ingestion module 230 receives the multimodal data and the unified tensor representation module 240 generates the tensor entry T[A, t₀]. The feature vector f(A, t₀) contains normalized values across clinical constructs: depressed mood = 0.58 (derived from PHQ-9 items 1-2), anhedonia = 0.50 (PHQ-9 items 3-4), sleep disturbance = 0.64 (derived from both PHQ-9 item 3 and actigraphy sleep efficiency), concentration = 0.45 (PHQ-9 item 7), anxiety severity = 0.55 (GAD-7 composite), and psychomotor change = 0.30 (PHQ-9 item 8). The missingness indicator vector m(A, t₀) encodes m=1 for EEG features, galvanic skin response features, and respiratory cadence features, as these modalities are not available for Patient A. The confidence weight vector w(A, t₀) assigns: PHQ-9-derived features w=0.92 (validated instrument with known psychometric properties), actigraphy-derived features w=0.85 (objective sensor data with moderate artifact rate), and clinician note NLP-extracted features w=0.71 (automated extraction with moderate confidence). Consent-gating flags g(A, t₀) are set to 1 (permitted) for all available modalities based on Patient A's documented consent.

Prior Construction. Because Patient A has 8 prior observations, the ANAF engine 250 constructs an individualized prior by fitting a distributional model to Patient A's historical state trajectory. The fitted prior for the depression severity dimension has mean μ=0.52 and variance σ²=0.04, reflecting Patient A's historical range. By contrast, for a new patient with no prior observations, the engine would initialize with a population-level prior having mean μ=0.40 and inflated variance σ²=0.16 (four times the individualized variance), such that the initial conservatism is proportional to the system's uncertainty about the specific patient. For a patient with only 2 prior observations, the engine would blend the limited patient data (weight=0.25) with the demographically stratified population prior (weight=0.75), yielding an intermediate prior.

Trait-State Decomposition. The ANAF engine 250 decomposes Patient A's depression severity signal into a trait component and a state component. Over Patient A's 14-week history, the trait component has stabilized at a value of 0.50, representing Patient A's chronic baseline depression level with sertraline treatment. The current observation at t₀ yields a state component of +0.08 (representing the acute deviation from the trait baseline: 0.58 - 0.50 = 0.08). Consider a subsequent observation at time t₁, two weeks later, in which Patient A's PHQ-9 score increases to 18 (normalized depression severity = 0.72). The trait-state decomposition identifies: trait component = 0.50 (unchanged, as traits are updated on longer timescales with higher regularization), and state component = +0.22 (acute deviation: 0.72 - 0.50 = 0.22). This +0.22 acute deviation exceeds a configurable clinical-significance threshold of 0.15, triggering a clinically significant acute deterioration alert. Notably, a conventional threshold-based CDS system using a fixed population severity cutoff of, for example, 0.80 (corresponding to a PHQ-9 score of approximately 20) would not generate an alert because Patient A's absolute score of 0.72 remains below the population threshold. The ANAF engine detects the deterioration because it evaluates change relative to the individual's own baseline trajectory rather than against a fixed population cutoff.

Bayesian State Update. At time t₁, the ANAF engine 250 performs a Bayesian state update for the depression severity dimension. The prior distribution from the individualized prior construction has mean μₚ=0.55 and variance σ²ₚ=0.035 (reflecting the prior state plus drift modeling). The observation likelihood from the PHQ-9 at t₁ suggests depression severity of 0.72, with the observation likelihood modulated by the PHQ-9 confidence weight of 0.92, yielding an effective observation variance of σ²ₒ=0.03/0.92=0.033. The posterior distribution is computed as proportional to the product of the prior and the observation likelihood, yielding a posterior mean of μₚ₀ₛₜ=0.64 and posterior variance σ²ₚ₀ₛₜ=0.017. The posterior mean shifts toward the observation but is tempered by the prior, and the posterior variance is reduced relative to both the prior and observation variances, reflecting increased confidence from combining two sources of evidence.

Uncertainty Decomposition. The uncertainty decomposition engine 650 decomposes the total posterior variance for each dimension into epistemic and aleatoric components. For the sleep disturbance dimension, total uncertainty = 0.15, decomposed as: epistemic uncertainty = 0.11 (attributable to the fact that actigraphy data spans only 3 nights, providing limited observational support) and aleatoric variance = 0.04 (attributable to intrinsic night-to-night sleep variability). The system annotates the clinician-facing output with the information that additional actigraphy data collection (e.g., 7 or more nights) would be expected to reduce the epistemic component from 0.11 to approximately 0.04, while the aleatoric component of 0.04 would remain unchanged because it reflects inherent variability. For the depression severity dimension at t₁, total uncertainty = 0.017, decomposed as: epistemic = 0.007 and aleatoric = 0.010, indicating that the depression estimate is well-supported by data and that most remaining uncertainty is intrinsic.

Cross-Pillar Propagation. The increase in Patient A's depression severity from 0.55 to 0.64 in the PS-TIPSV triggers cross-pillar coupling effects via the coupling matrix. The Risk State Vector (RiskSV) updates its depression-risk dimension, resulting in the RiskSV composite risk estimate increasing from 0.35 to 0.52. Because the RiskSV now exceeds a configurable destabilization-risk gate threshold of 0.45, the treatment sequencer's feasible action space is restricted: the full action space of 12 candidate interventions is reduced to 7 candidates by excluding high-intensity interventions (e.g., intensive trauma-focused processing, rapid medication titration) that carry elevated destabilization risk. Separately, the elevated epistemic uncertainty in the sleep dimension (ε=0.11) propagates through the coupling matrix to the medication parameter modeling pillar, where the safety-constrained optimizer 920 computes an uncertainty-adjusted action radius. With epistemic uncertainty of 0.11 against a baseline action radius of 3 candidate dose changes, the radius shrinks to permit only 1 candidate action: maintenance of the current sertraline dose at 100 mg with increased sleep monitoring. Under this condition, the system does not suggest dose increases or medication additions because the epistemic uncertainty about the patient's sleep state is too high to justify aggressive pharmacologic intervention.

Volatility Fingerprint Matching. At time t₁, the ANAF engine 250 computes volatility estimates across state-vector dimensions over a 4-week window. The computed volatility fingerprint shows: sleep volatility = 0.18 (elevated, normal range 0.05-0.10), mood volatility = 0.15 (elevated, normal range 0.04-0.08), concentration volatility = 0.12 (moderately elevated, normal range 0.03-0.07), anxiety volatility = 0.09 (within normal range), and psychomotor volatility = 0.06 (within normal range). The pattern of simultaneous elevation in sleep, mood, and concentration volatility with stable anxiety and psychomotor dimensions is compared against the library of learned destabilization signatures. Signature DS-007 ("sleep-mood-cognitive destabilization cluster") produces a similarity score of 0.87, exceeding the configurable threshold of 0.75. The system generates a destabilization alert with priority = HIGH, matched signature = DS-007, and attribution identifying sleep volatility (contribution weight 0.42), mood volatility (0.35), and concentration volatility (0.23) as the primary drivers.

Clinician Artifact. The clinician interface module 220 renders the following decision-support artifact for clinician review. The diagnostic section presents ranked diagnostic differentials: (1) Major Depressive Disorder, recurrent, moderate severity — posterior probability 0.72, confidence interval [0.61, 0.83]; (2) Generalized Anxiety Disorder — posterior probability 0.58, CI [0.44, 0.72]; (3) Mixed Anxiety-Depressive presentation — posterior probability 0.35, CI [0.22, 0.48]. The uncertainty annotations indicate that the depression estimate carries primarily aleatoric uncertainty (well-supported by data but inherently variable), while the anxiety estimate carries elevated epistemic uncertainty (additional GAD-7 administration recommended within 1 week). The treatment section recommends: (a) maintain current sertraline 100 mg (medication action restricted by uncertainty-driven action-space shrinking), (b) increase sleep monitoring via continued actigraphy (epistemic uncertainty reduction opportunity), (c) schedule behavioral activation session within 5 days (permitted within the restricted action space). The constraint-satisfaction summary confirms: destabilization-risk gate satisfied (risk below crisis threshold), cognitive-load ceiling satisfied (proposed interventions within estimated capacity), and scope-of-practice confirmed (all proposed interventions within assigned clinician's competencies). The alert panel highlights the destabilization-pattern match with DS-007 and recommends increased monitoring cadence from biweekly to weekly. No computational output constitutes a final clinical action; the clinician reviews the artifact and provides an approval, modification, deferral, or rejection disposition.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more subprocessing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated for the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A computer-implemented method for clinician-governed, uncertainty-bounded, closed-loop psychiatric clinical decision support, the computer-implemented method executed by one or more processors and comprising:
accessing multimodal patient data for a patient from a plurality of data sources;
generating a tensor representation synthesizing the multimodal data into a unified temporally indexed structure comprising, for each observation, feature vectors, missingness encodings, confidence weights, source provenance metadata, and consent-gating flags;
generating one or more probabilistic state vectors encoding psychiatric status across a plurality of clinical dimensions, wherein generating the one or more probabilistic state vectors comprises decomposing the multimodal patient data using a hierarchical latent variable model, and computing, for each state vector, mean estimates and associated uncertainty parameters;
computing a longitudinal trajectory over the one or more probabilistic state vectors, the longitudinal trajectory comprising at least one of: drift, acceleration, and volatility estimates for each state-vector dimension;
generating a treatment plan through safety-constrained optimization on the longitudinal trajectory, wherein the optimization is subject to hard safety constraints comprising at least one of contraindication exclusion, destabilization-risk gating, or cognitive-load budgeting, wherein increased uncertainty in the one or more probabilistic state vectors algorithmically shrinks action space of the optimization;
causing presentation of the treatment plan on a clinician-facing user interface to obtain clinician approval;
receiving, via the clinician-facing user interface, clinician approval of the treatment plan;
upon receipt of clinician approval, causing execution of the treatment plan by presenting one or more actions of the treatment plan on a patient-facing user interface.

2. The computer-implemented method of claim 1, wherein the plurality of data sources comprises electronic health records, clinician-entered assessments, patient-reported outcome measures, behavioral engagement telemetry, biometric sensor streams, pharmacy and medication data, administrative and operational data, or some combination thereof.

3. The computer-implemented method of claim 1, wherein generating the one or more probabilistic state vectors:
comprises constructing individualized prior distributions by:
fitting a distributional model to a state trajectory derived from historical patient data.

4. The computer-implemented method of claim 1, wherein generating the one or more probabilistic state vectors:
comprises constructing individualized prior distributions by:
initializing with a population-level prior having a covariance inflated by a configurable factor to reflect uncertainty about the patient.

5. The computer-implemented method of claim 1, wherein generating the one or more probabilistic state vectors further comprises performing Bayesian state updates upon receipt of new data, wherein a posterior distribution is computed as proportional to a product of a prior distribution and an observation likelihood, and wherein the observation likelihood is modulated by confidence weights such that higher-confidence observations contribute more strongly to the posterior update.

6. The computer-implemented method of claim 5, wherein features with indicators representing that the feature was not observed are excluded from the observation likelihood computation, such that dimensions associated with missing features have their uncertainty increase over time through prior dynamics alone.

7. The computer-implemented method of claim 1, wherein computing longitudinal trajectory statistics further comprises:
computing volatility fingerprints, each volatility fingerprint comprising a pattern of variance changes across a plurality of state-vector dimensions; and
comparing said volatility fingerprints against a library of learned destabilization signatures.

8. The computer-implemented method of claim 1, wherein computing the longitudinal trajectory further comprises:
applying Bayesian online change-point detection that maintains a posterior distribution over time since a recent change point and flags a dimension as having undergone a statistically significant shift when a posterior probability of the recent change point exceeds a configurable threshold.

9. The computer-implemented method of claim 1, further comprising:
computing safety-constrained policy outputs by applying conservative gating when propagated uncertainty exceeds configurable thresholds, said conservative gating comprising at least one of:
reducing a magnitude of recommended changes,
increasing recommended monitoring frequency,
narrowing a set of permissible actions,
withholding output pending clinician review, and
annotating the output with an explicit low-confidence indicator identifying whether elevated uncertainty is primarily epistemic or aleatoric.

10. The computer-implemented method of claim 1, wherein generating the one or more probabilistic state vectors is performed using an inference method selected from the group consisting of: variational inference with a parametric posterior approximation; sequential Monte Carlo with weighted particle representation; extended Kalman filtering adapted for psychiatric state spaces; and unscented Kalman filtering.

11. The computer-implemented method of claim 1, wherein the unified temporally indexed representation is implemented as a sparse data structure storing observed entries, configured for incremental update upon receipt of new data without full re-computation and deterministic reconstruction of any prior data state from logged observations.

12. The computer-implemented method of claim 1, further comprising:
evaluating concordance across data modalities for each clinical construct represented in the state vectors, and
when statistically significant discordance is detected between modalities, increasing the uncertainty parameters of affected state-vector dimensions.

13. The computer-implemented method of claim 12, wherein clinician-review artifacts include discordance annotations identifying which modalities disagree and a magnitude of disagreement for each affected clinical construct.

14. The computer-implemented method of claim 1, wherein further comprising:
compiling outputs into operationally executable workflow artifacts.

15. The computer-implemented method of claim 14, wherein compiling the outputs into operationally executable workflow artifacts further comprises routing the patient among a plurality of care tiers modeled as a directed graph.

16. The computer-implemented method of claim 14, wherein compiling the outputs into operationally executable workflow artifacts further comprises decomposing a clinician-approved care plan into a directed acyclic graph of atomic executable tasks, each task comprising a task type and a scheduled execution time or trigger condition.

17. The computer-implemented method of claim 1, further comprising:
executing a crisis response protocol modeled as a state machine comprising a sequence of escalation states with transition rules, wherein each state transition carries a service-level-agreement timer.

18. The computer-implemented method of claim 1, wherein the one or more probabilistic state vectors include a diagnostic state vector, and wherein generating the diagnostic state vector comprises:
encoding psychiatric diagnostic criteria as a probabilistic constraint graph in which each criterion is associated with an activation probability derived from relevant state-vector dimensions; and
computing a posterior probability distribution over the number of criteria to yield a graded diagnostic confidence.

19. The computer-implemented method of claim 1, wherein the one or more probabilistic state vectors include a risk state vector, and wherein generating the risk state vector comprises:
computing multi-horizon probabilistic risk trajectories by projecting a risk state vector forward through a learned state-transition model, the trajectories comprising probability distributions over future risk states at a plurality of time horizons.

20. The computer-implemented method of claim 1, wherein the one or more probabilistic state vectors include a latent pharmacologic state vector, and wherein generating the latent pharmacologic state vector comprises:
modeling the latent pharmacologic state vector against stability-flexibility balance, adverse-event susceptibility, tolerance dynamics, or some combination thereof as time-varying probabilistic estimates.

21. The computer-implemented method of claim 1, wherein generating the treatment plan comprises:
selecting a set of candidate medication actions from a parameterized control-action library; and
applying constrained optimization subject to hard constraints, with uncertainty-driven action-space shrinking that restricts the set of candidate medication actions as uncertainty parameters increase.

22. The computer-implemented method of claim 21, wherein the uncertainty-driven action-space shrinking defines an uncertainty-adjusted action radius that is a monotonically decreasing function of epistemic uncertainty.

23. The computer-implemented method of claim 21, wherein generating the treatment plan further comprises:
identifying high-sensitivity therapeutic windows from the longitudinal trajectory; and
restricting medication adjustments during the high-sensitivity therapeutic windows.

24. The computer-implemented method of claim 1, wherein the one or more probabilistic state vectors comprise a plurality of linked state vectors across distinct clinical domains, and wherein the method further comprises:
generating a cross-pillar coupling matrix defining directional relationships between state vectors, such that a change in a source state vector causes conditional prior updates in coupled target state vectors.

25. The computer-implemented method of claim 24, wherein the cross-pillar coupling matrix couples elevated destabilization risk in a risk state vector with restriction in permissible treatment intensity in a treatment sequencing computation or with restriction in permissible medication action space in a pharmacologic parameter computation.

26. The computer-implemented method of claim 24, wherein the cross-pillar coupling matrix couples increased diagnostic uncertainty in a diagnostic state vector with decreases in permissible medication action space in a pharmacologic parameter computation.

27. The computer-implemented method of claim 24, wherein the cross-pillar coupling matrix couples medication volatility indicators in a pharmacologic state vector feed back to a risk state vector as input features.

28. The computer-implemented method of claim 24, wherein the cross-pillar coupling matrix couples operational feasibility constraints in an operational state vector to bound treatment and medication recommendations.

29. A non-transitory computer-readable storage medium storing instructions for clinician-governed, uncertainty-bounded, closed-loop psychiatric clinical decision support, the instructions that, when executed, cause one or more processors to perform operations comprising:
accessing multimodal patient data for a patient from a plurality of data sources;
generating a tensor representation synthesizing the multimodal data into a unified temporally indexed structure comprising, for each observation, feature vectors, missingness encodings, confidence weights, source provenance metadata, and consent-gating flags;
generating one or more probabilistic state vectors encoding psychiatric status across a plurality of clinical dimensions, wherein generating the one or more probabilistic state vectors comprises decomposing the multimodal patient data using a hierarchical latent variable model, and computing, for each state vector, mean estimates and associated uncertainty parameters;
computing a longitudinal trajectory over the probabilistic state vectors, the longitudinal trajectory comprising at least one of: drift, acceleration, and volatility estimates for each state-vector dimension;
generating a treatment plan through safety-constrained optimization on the longitudinal trajectory, wherein the optimization is subject to hard safety constraints comprising at least one of contraindication exclusion, destabilization-risk gating, or cognitive-load budgeting, wherein increased uncertainty in the one or more probabilistic state vectors algorithmically shrinks action space of the optimization;
causing presentation of the treatment plan on a clinician-facing user interface to obtain clinician approval;
receiving, via the clinician-facing user interface, clinician approval of the treatment plan;
upon receipt of clinician approval, causing execution of the treatment plan by presenting one or more actions of the treatment plan on a patient-facing user interface.

30. A system for clinician-governed, uncertainty-bounded, closed-loop psychiatric clinical decision support, the system comprising:
one or more processors; and
a non-transitory computer-readable storage medium storing instructions that, when executed, cause the one or more processors to perform operations comprising:
accessing multimodal patient data for a patient from a plurality of data sources;
generating a tensor representation synthesizing the multimodal data into a unified temporally indexed structure comprising, for each observation, feature vectors, missingness encodings, confidence weights, source provenance metadata, and consent-gating flags;
generating one or more probabilistic state vectors encoding psychiatric status across a plurality of clinical dimensions, wherein generating the one or more probabilistic state vectors comprises decomposing the multimodal patient data using a hierarchical latent variable model, and computing, for each state vector, mean estimates and associated uncertainty parameters;
computing a longitudinal trajectory over the probabilistic state vectors, the longitudinal trajectory comprising at least one of: drift, acceleration, and volatility estimates for each state-vector dimension;
generating a treatment plan through safety-constrained optimization on the longitudinal trajectory, wherein the optimization is subject to hard safety constraints comprising at least one of contraindication exclusion, destabilization-risk gating, or cognitive-load budgeting, wherein increased uncertainty in the one or more probabilistic state vectors algorithmically shrinks action space of the optimization;
causing presentation of the treatment plan on a clinician-facing user interface to obtain clinician approval;
receiving, via the clinician-facing user interface, clinician approval of the treatment plan;
upon receipt of clinician approval, causing execution of the treatment plan by presenting one or more actions of the treatment plan on a patient-facing user interface.
